(19) United States
(12) Patent Application Publication
(10) Pub. No.: US 2026/0060347 A1
(43) Pub. Date: Mar. 5, 2026

(54) SYSTEM AND METHOD FOR MANAGING OPERATING CONDITION PARAMETERS IN MEDICAL DEVICES

(71) Applicants: Mark T. McBride, Coronado, CA (US); Glenn Butler, Holliston, MA (US); John Roughneen, Dennis, MA (US)

(72) Inventors: Mark T. McBride, Coronado, CA (US); Glenn Butler, Holliston, MA (US); John Roughneen, Dennis, MA (US)

(21) Appl. No.: 19/309,711

(22) Filed: Aug. 26, 2025

Related U.S. Application Data
(60) Provisional application No. 63/687,364, filed on Aug. 27, 2024.

(51) Int. Cl. A41D 27/28; A41D 1/00; A41D 13/11; A41D 13/12; A61M 16/00

(52) U.S. Cl. CPC A41D 27/28; A41D 1/002; A41D 13/1153; A41D 13/1218; A61M 16/0003; A61M 16/0051; A41D 2600/00; A61M 2016/0027; A61M 2205/502; A61M 2205/80

(57) ABSTRACT

Systems, methods, and other embodiments for managing operating condition parameters in medical devices. The system would allow the user to set operating condition parameter preferences within a surgical hood while the user is wearing the surgical hood during a surgical procedure, wherein the operating condition parameter preferences include, but are not limited to, threshold levels of carbon dioxide (CO₂), temperature, humidity, oxygen (O₂), volatile organic compounds (VOCs), and/or air pressure. The system would then keep track of the operating condition parameters (or air quality) within the hood while the user is wearing the hood during the surgical procedure. The system would also provide feedback to the user regarding the operating condition parameters of the system while the system is being used so that the user is aware of the threshold levels of carbon dioxide (CO₂), temperature, humidity, oxygen (O₂), VOCs, and/or air pressure conditions within the medical device.

### CROSS-REFERENCE TO RELATED APPLICATION

This application claims benefit of U.S. Patent Application 63/687,364, filed on Aug. 27, 2024, the disclosure of which is hereby incorporated by reference in its entirety to provide continuity of disclosure to the extent such a disclosure is not inconsistent with the disclosure herein.

### FIELD OF THE INVENTION

The present invention is generally related to a system and method for managing operating condition parameters in medical devices. The system would allow the user to set operating condition parameter preferences within a medical device such as a surgical hood while the user is wearing the surgical hood during a surgical procedure, wherein the operating condition parameter preferences include, but are not limited to, threshold levels of carbon dioxide (CO₂), temperature, humidity, oxygen (O₂), volatile organic compounds (VOCs), and/or air pressure. The system would then keep track of the operating condition parameters (or air quality) within a medical device while the user is wearing the surgical hood during the surgical procedure. The system would also provide feedback to the user regarding the operating condition parameters of the system while the system is being used so that the user is aware of the threshold levels of carbon dioxide (CO₂), temperature, humidity, oxygen (O₂), VOCs, and/or air pressure conditions within the medical device.

For example, if the medical device is a surgical hood having a ventilation system and the user is wearing the surgical hood, it would be desirable for the user to be made aware of the operating parameter conditions within the surgical hood (e.g., the levels of carbon dioxide (CO₂), temperature, humidity, oxygen (O₂), VOCs, and/or air pressure) while the ventilation system is being operated. In this manner, if one of the operating parameter conditions within the hood exceeds a pre-determined threshold as set by the user and stored in the user preferences of the system, the medical device could then interact with the ventilation system in order to attempt to correct the operating parameter condition that has exceeded its pre-determined threshold within the hood. Furthermore, the system could be equipped with an alarm (or alarms) such as an audible and haptic (vibrate) alarm that will alert the user that an operating parameter condition has exceeded the pre-determined threshold and/or that the system has attempted to correct the operating parameter condition that has exceeded its pre-determined threshold, but the system has not been successful in correcting the operating parameter condition that has exceeded its pre-determined threshold. In this latter instance, the user may need to remove the medical device and have maintenance performed on the medical device. Finally, the system would be able to collect information about the operating parameter conditions of the medical devices for user edification and/or product improvement.

### BACKGROUND OF THE INVENTION

Prior to the present invention, as set forth in general terms above and more specifically below, it is known to employ various types of hoods and one-piece gowns or togas for use during medical procedures. The gowns are designed to cover the wearer completely and sterilely when they are attached to the hood. Currently, a helmet or other similar head support structure is donned by the wearer and the one-piece gown and the hood are conventionally attached to the helmet or other similar head support structure. Furthermore, it is known to provide a ventilation system that is also attached to the helmet or other similar head support structure or attached to the wearer.

When the hood and gown are donned by the wearer, it is important that the hood and gown completely and sterilely cover the wearer, as discussed above. In this manner, a closed area is created around the wearer's head and neck areas. Consequently, ventilation must be provided within this closed area so that the wearer can wear the hood and gown and still be able to properly perform the medical procedure without having to worry about encountering high levels of carbon dioxide (CO₂), temperature, humidity, oxygen (O₂), VOCs, and/or air pressure within the closed area.

Furthermore, it is known that medical devices need to be properly installed and maintained to function properly. A medical device that is not properly installed or maintained may fail at a very inopportune time, which could lead to a serious incident during a medical operation or procedure. For example, if the medical device is not properly installed or maintained, the wearer may experience high levels of carbon dioxide (CO₂), temperature, humidity, oxygen (O₂), VOCs, and/or air pressure within the closed area which adversely affect the wearer.

It is a purpose of this invention to fulfill these and other needs in the medical device art in a manner more apparent to the skilled artisan once given the following disclosure.

The preferred system and method for managing operating condition parameters in medical devices, according to various embodiments of the present invention, offers the following advantages: ease of use; the ability to keep track of operating condition parameters in medical devices; the ability to automatically correct the operating condition parameter in the medical device without user intervention, if needed; the ability to provide feedback regarding the operating condition parameters in the medical device for wearer edification, preventative maintenance, and or product improvement; the ability to allow the user to pre-set levels of operating conditions within the hood; and the ability to provide audible and haptic (vibrate) alerts. In fact, in many of the preferred embodiments, these advantages are optimized to an extent that is considerably higher than heretofore achieved in prior, known systems and methods for managing operating condition parameters in medical devices.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic, isometric view of a helmetless support for use with surgical hoods and gowns, according to one embodiment of the present invention;

FIG. 2 is a schematic, isometric view of a ventilation system for use with surgical hoods and gowns, constructed according to an embodiment of the present invention;

FIG. 3 is a schematic, side view of the ventilation system for use with surgical hoods and gowns illustrating the adjustable face vent air flow levers, constructed according to an embodiment of the present invention;

FIG. 4 is a cut-away view, taken along lines 4-4 of FIG. 3 of the ventilation system for use with surgical hoods and gowns illustrating the adjustable face vent air flow levers, constructed according to an embodiment of the present invention;

FIG. 5 is a schematic, isometric, back view of the ventilation system for use with surgical hoods and gowns with the back cover removed illustrating the power module, constructed according to an embodiment of the present invention;

FIG. 6 is an isometric, back view of the ventilation system for use with surgical hoods and gowns with the back cover removed illustrating the printed circuit board (PCB) module, constructed according to an embodiment of the present invention;

FIG. 7 is a schematic illustration of the ventilation system for use with surgical hoods and gowns illustrating the operating condition parameter measurement device being located adjacent to the wearer in one embodiment, constructed according to an embodiment of the present invention;

FIG. 8 illustrates one embodiment for a method for managing operating parameters in medical devices;

FIG. 9 is a schematic illustration of a system for managing operating parameters in medical devices, according to the present invention;

FIG. 10 is a schematic illustration of the ventilation system for use with surgical hoods and gowns being attached to a user,, according to the present invention;

FIG. 11 is a schematic illustration of a screen shot of the login and welcome page of the companion application as displayed on the mobile computing device configured with the application for use with the system for managing operating parameters in medical devices, according to the present invention;

FIG. 12 is a schematic illustration of a screen shot of the personalized user preferences page of the companion application as displayed on the mobile computing device configured with the application that are sent to the medical device, according to the present invention;

FIG. 13 is a schematic illustration of a screens shot of the application settings page of the companion application as displayed on the mobile computing device configured with the application for training voice commands for the application for use with the system for managing operating parameters in medical devices, according to the present invention;

FIG. 14 is a schematic illustration of a screen shot of the current operating conditions as displayed on the mobile computing device being used with the medical device, according to the present invention;

FIG. 15 is a schematic illustration of a screen shot an alert as displayed on the mobile computing device that an operating condition parameter threshold has been exceeded and the system for managing operating parameters in medical devices is correcting the operating condition, according to the present invention;

FIG. 16 is a schematic illustration of a screenshot of an alert as displayed o the mobile computing device that the operating condition has been corrected by the system for managing operating parameters in medical devices and the system has returned to normal operating conditions, according to the present invention;

FIG. 17 is a schematic illustration of the interaction between the companion application configured on the mobile computing device and the medical device, according to the present invention;

FIG. 18 is a schematic illustration of the system for managing operating parameters in medical devices interacting with the cloud and a web portal, according to the present invention; and

FIG. 19 illustrates an embodiment of a computing system configured with the example systems and/or methods disclosed.

### DETAILED DESCRIPTION OF THE PREFERRED

### Embodiments of the Invention

In order to address the shortcomings of the prior, known systems and methods for managing operating parameters in a medical device, it would be desirable to utilize a system and method for managing operating condition parameters in medical devices. The system would allow the user to set operating condition parameter preferences within a medical device such as a surgical hood while the user is wearing the surgical hood during a surgical procedure, wherein the operating condition parameter preferences include, but are not limited to, threshold levels of carbon dioxide (CO₂), temperature, humidity, oxygen (O₂), volatile organic compounds (VOCs), and/or air pressure within the surgical hood. The system would then keep track of the operating condition parameters (or air quality) within a medical device while the user is wearing the surgical hood during the surgical procedure. The system would provide feedback to the user regarding the operating condition parameters of the system while the medical device is being worn during a medical procedure so that the user is aware of the threshold levels of carbon dioxide (CO₂), temperature, humidity, oxygen (O₂), VOCs, and/or air pressure conditions within the medical device.

For example, if the medical device is a surgical hood having a ventilation system and the user is wearing the surgical hood, it would be desirable for the user to be made aware of the operating parameter conditions within the surgical hood (e.g., the levels of carbon dioxide (CO₂), temperature, humidity, oxygen (O₂), VOCs, and/or air pressure) while the ventilation system is being operated. In this manner, if one of the operating parameter conditions within the hood exceeds a pre-determined threshold as set by the user and stored in the user preferences of the system, the medical device could then interact with the ventilation system in order to attempt to correct the operating parameter condition that has exceeded its pre-determined threshold within the hood. Furthermore, the system could be equipped with an alarm (or alarms) such as an audible and haptic (vibrate) alarm that will alert the user that an operating parameter condition has exceeded the pre-determined threshold and/or that the system has attempted to correct the operating parameter condition that has exceeded its pre-determined threshold, but the system has not been successful in correcting the operating parameter condition that has exceeded its pre-determined threshold. In this latter instance, the user may need to remove the medical device and have maintenance performed on the medical device. Finally, the system would be able to collect information about the operating parameter conditions of the medical devices for user edification and/or product improvement.

A unique aspect of the present invention is that the pre-determined threshold value can be set according to, but not limited to, the following:
1. A value set by the user for comfort;
2. A value set by the hospital/system administrator policy;
3. A value based upon best practices; and/or
4. A value based upon compliance with Occupational Safety and Health Administration (OSHA) policies.

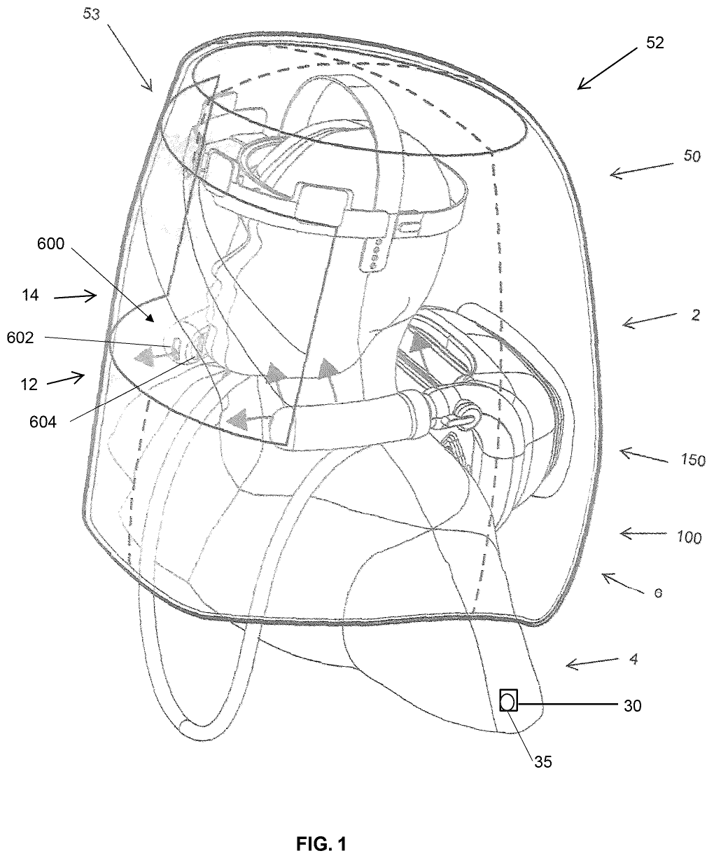

Referring now to FIG. 1-7, there is illustrated a helmetless support system 2 for use with surgical hoods and gowns. The helmetless support system 2 for use with surgical hoods and gowns can be used to support the one-piece surgical gown 4 and the surgical hood 14 without the need for the wearer 6 to wear a helmet. In this manner, one-piece surgical gown 4 and the surgical hood 14 completely and sterilely covers the head, neck, and torso of the wearer 6 when donned by the wearer 6. Also, the one-piece surgical gown 4 and the surgical hood 14 includes a clear faceplate 12. The helmetless support 2 further includes a helmetless surgical hood and gown support having a flexible headband 52 with attached lightweight front offsets 53 in front that can be releasably attached to the faceplate 12. Furthermore, the front offsets 53 are used to provide air circulation around head of the wearer 6.

### Helmetless Surgical Hood and Gown Support

As shown in FIG. 1, helmetless support 2 for use with surgical hoods and gowns includes, in part, surgical gown 4, wearer 6, gown wireless identification system 30, helmetless surgical hood and gown support 50, and operating condition parameter sensor 602. It is to be understood that surgical gown 4 is constructed of any suitable, durable, medical grade material. It is to be further understood that the surgical gown 4 is to be constructed into a one-piece design that will completely and sterilely cover the wearer when attached to the hood 14. Finally, it is to be understood that gown wireless identification system 30 is a conventional wireless identification system such as an RFID tag 35 that can be conventionally attached to the surgical gown 4.

With respect to helmetless surgical hood and gown support 50, helmetless surgical hood and gown support 50 includes, in part, flexible, adjustable band 52 and front offsets 53. Preferably, flexible band 52 is constructed of any suitable, durable, flexible, medical grade material. An important feature of flexible band 52 being that it comfortably fits around the head of the wearer 6, but still is capable of securely holding surgical gown 4 and surgical hood 14 once the surgical gown 4 and surgical hood 14 have been attached to helmetless surgical hood and gown support 50 and then placed over the wearer, as will be discussed in greater detail later. In particular, it is important that flexible band 52 be able to securely hold hood 14 off of the head of wearer 6 and allow the air to flow around the head of wearer 6, as will be discussed in greater detail later.

### Helmetless Surgical Hood and Gown Ventilation System

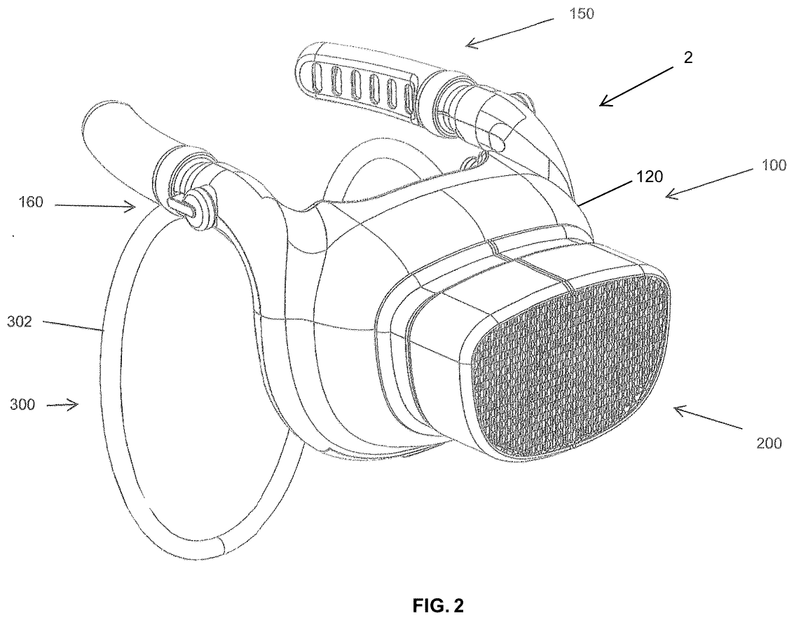

Referring now to FIGS. 2-7, there is illustrated a ventilation system 100 for use with helmetless support system 2. The ventilation system 100 is constructed such that the fan speed can be controlled by the wearer 6 and/or the system 2 once the gown 4 and hood 14 have been donned. A face vent module 150 is used as a “yoke” to support the ventilation system 100 on the shoulders of the wearer 6 (FIG. 1). Finally, the wearer 6 can control the output from each of the various output apertures in face vent module 150 and neck vent module 350 in the ventilation system 100, as will be discussed in greater detail later.

As shown in FIGS. 2-7, helmetless support system 2 for use with surgical hoods and gowns having ventilation system 100 includes, in part, protective casing 120, face vent module 150, air filtration module 200, power module 250, yoke module 300, neck vent module 350, air flow generation module 450, printed circuit board (PCB) module 500, and the operating parameter measurement assembly 600.

A unique aspect of the present invention is the location of the ventilation system 100 with respect to the surgical gown 4 and surgical hood 14. As shown in FIG. 1, the ventilation system 100 is almost completely located inside of the surgical gown 4 and surgical hood 14.

### Protective Casing 120

With respect to protective casing 120, protective casing 120, preferably, is constructed of any suitable, durable, high strength, shock resistant, UV resistant, medical grade polymeric material. It is to be understood that protective casing 120 is used to encase ventilation system 100 to provide protection for air filtration module 200, power module 250, neck vent module 350, air flow generation module 450, and printed circuit board (PCB) module 500.

### Face Vent Module 150

Regarding face vent module 150, as shown in FIGS. 2-6, face vent module 150, includes, in part, removable face vents 152, face vent openings 154, face vent connectors 156, face vent adaptors 158, face vent air flow adjustors 160, and face vent air flow adjuster lever 162. Preferably, face vents 152 and face vent connectors 156 are constructed as a single-piece construction and are constructed of any suitable, durable, lightweight, medical grade, and washable material. Also, face vent openings 154 are formed in removable face vents 152 by conventional techniques such as forming, stamping, molding, or the like. Face vent adaptors 158, preferably, are constructed of any suitable, durable, high strength, medical grade material and are permanently connected to protective casing 120 near face vent air flow adjustors 160 and face vent air flow adjuster levers 162. Finally, face vent air flow adjustors 160 and face vent air flow adjuster lever 162, preferably, are constructed of any suitable, durable, high strength, medical grade material.

A unique aspect of the present invention is the use of removable face vents 152. In particular, removable face vents 152 are constructed in such a manner that allows the removable face vents 152 to be easily removed from the face vent adaptors 158 so that the removable face vents 152 can be cleaned, disinfected, and sanitized prior to the next usage of the helmetless support 2 for use with surgical hoods and gowns having ventilation system 100. Once the removable face vents 152 have been cleaned, disinfected, and sanitized, the removable face vents 152 can be easily slid onto the face vent adaptors 158 by locating the face vent connectors 156 on the face vent adaptors 158.

A further unique aspect of the present invention is the use of face vent air flow adjustors 160 and face vent air flow adjuster lever 162. In particular, the wearer 6 can adjust the amount of air flow that is being emitted out of the removable face vents 152 through the use of vent air flow adjustor 160 and face vent air flow adjuster lever 162. In this manner, the wearer 6 can conventionally manipulate face vent air flow adjuster lever 162 so that the amount of air flow is adjusted. For example, the wearer 6 may push/pull the face vent air flow adjuster lever 162 upwards which will cause the amount of air flow being emitted out of the removable face vents 152 to be reduced. Conversely, the wearer 6 may push/pull the face vent air flow adjuster lever 162 downwards which will cause the amount of air flow being emitted out of the removable face vents 152 to be increased.

### Air Filtration Module 200

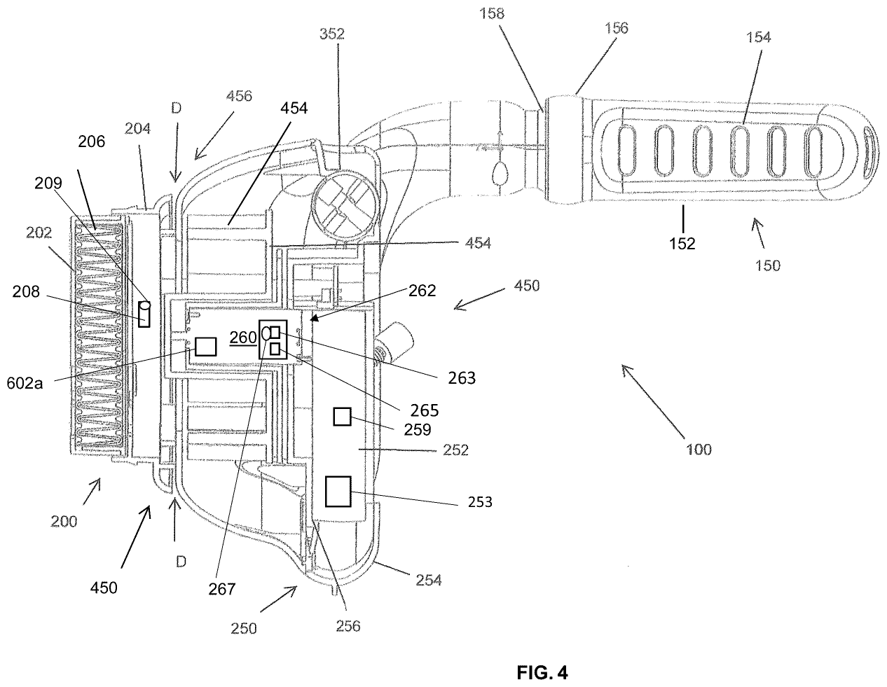

With respect to air filtration module 200, as shown in FIG. 4, air filtration module 200, includes, in part, air filter 202, air filtration adaptor 204, filter casing 206, and air filtration module wireless identification system 208. Preferably, air filter 202 is a HEPA (or ULPA) air filter that is located within filter casing 206. Preferably, filter casing 206 is constructed of any suitable, durable, high strength, medical grade material. Preferably, air filtration adaptor 204 is conventionally formed on protective casing 120. Finally, it is to be understood that air filtration module wireless identification system 208 is a conventional wireless identification system such as an RFID tag 209 that can be conventionally attached to or electrically connected to the air filter 202, as will be discussed in greater detail later.

A unique aspect of the present invention is the use of air filtration module 200. In particular, air filtration module 200 can be used to filter out air borne contaminants so that they do not enter into the surgical hood 14 and surgical gown 4. As discussed above, only air filter 202 extends outside of the surgical hood 14 (FIG. 1). In this manner, only air going through the air filtration module 200 will be allowed to enter the surgical hood 14 and surgical gown 4. Also, the air filter 202 can be easily removed and replaced. For example, wearer 6 can simply remove the air filter 202 and the filter casing 206 from the air filtration adaptor 204. The wearer 6 can then replace the used air filter 202 and filter casing 206 with a new air filter 202 and filter casing 206 by simply placing the new air filter 202 and filter casing 206 onto the air filtration adaptor 204. It is to be understood that the air filter 202 and filter casing 206 can be retained on the air filtration adaptor 204 by a snap fit, a threaded connection, a bayonet connection, a slidable connection or the like, as will be discussed in greater detail later.

### Power Module 250

Regarding power module 250, as shown in FIGS. 4-6, power module 250, includes, in part, battery 252, battery sensor 253, battery doors 254, battery lock 256, wireless power module identification system 258, motor sensor/wireless identification system 262, tachometer 263, and a printed control board 265. Preferably, battery 252 is a conventional, rechargeable battery such as a lithium-ion battery or the like that can provide sufficient power to air flow generation module 450, printed circuit board (PCB) module 500, and operating condition parameter measurement assembly 600 for an extended period of time such as 6-8 hours. Battery sensor 253 is a conventional sensor that can be used to monitor the operation of battery 252 to ensure that battery 252 is operating properly. Also, battery doors 254, preferably are constructed of any suitable, durable, high strength, medical grade material. It is to be understood that wireless power module identification system 258 is a conventional wireless identification system such as an RFID tag 259 that can be conventionally attached to or electrically connected to the battery 252, as will be discussed in greater detail later. It is to be understood that instead of an RFID tag 259, the battery 252 may include a serial number that can be conventionally read/detected using a hardware data line that is configured to be used with the battery 252 through the wireless power module identification system 258. It is to be further understood that motor sensor/wireless identification system 262 includes a conventional printed circuit board 265, a tachometer 263, a RFID tag 267 that can be conventionally attached to or electronically connected to the fan motor 260, as will be discussed in greater detail later. Furthermore, printed circuit board 265 is configured with an algorithm that is used to determine the operating speed of the fan motor 260 based on one or more of the previously discussed pre-determined thresholds. Finally, in one embodiment, the algorithm could be updated with information uploaded onto the printed circuit board 265 through an interaction with processor 2002 (FIG. 20) either manually or automatically based upon the desired interval in which such information is to be uploaded onto the printed circuit board 265.

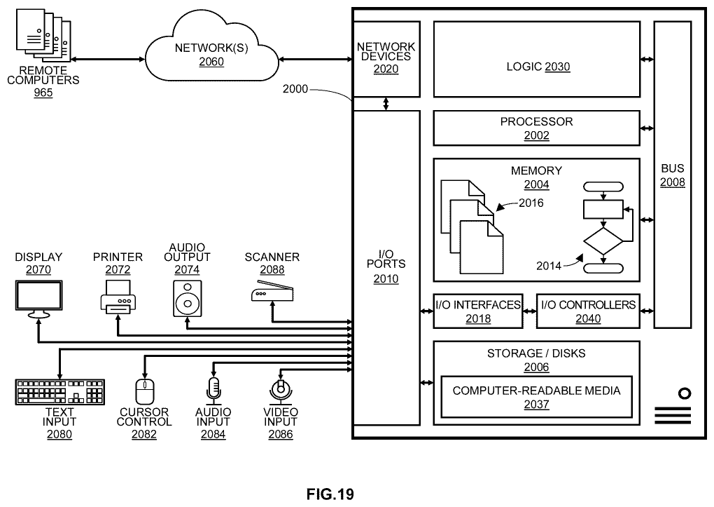

A unique aspect of the present invention is the use of battery sensor 253. In particular, battery sensor 253 can be used to monitor the cycles and voltages of battery 250 so that it can be determined when the battery 250 needs to be replaced. Also, the battery sensor 253 can be used in conjunction with the processor 2002 (FIG. 19) and printed circuit board (PCB) module 500 to utilize the information from battery sensor 253 in order to alert the user that the battery 250 is not functioning properly or to simply place an order for a replacement for battery 250.

Another unique aspect of the present invention is the use of battery doors 254. Battery doors 254 are conventionally connected to protective casing 120 so that battery doors 254 can swing (or pivot) open so that battery 252 can be easily installed into power module 250 or removed from power module 250. In particular, the wearer 6 can remove battery 252 from power module 250 by opening battery doors 254 and removing battery 252 from power module 250. The battery 252 can then be placed on a conventional battery charger (not shown). Once battery 252 has been fully charged, the wearer 6 can then remove the battery 252 from the battery charger, open the battery doors 254, and slide the battery 252 into the power module 250 so that the battery 252 is securely retained within the power module 250. The wearer 6 then closes the battery doors 254 so that the battery 252 is not exposed to the elements. It is to be understood that a conventional locking mechanism 256 can be used to lock the battery 252 in place in the power module 250 so that the battery 252 does not inadvertently come loose while the ventilation system 100 is being operated.

### Yoke Module 300

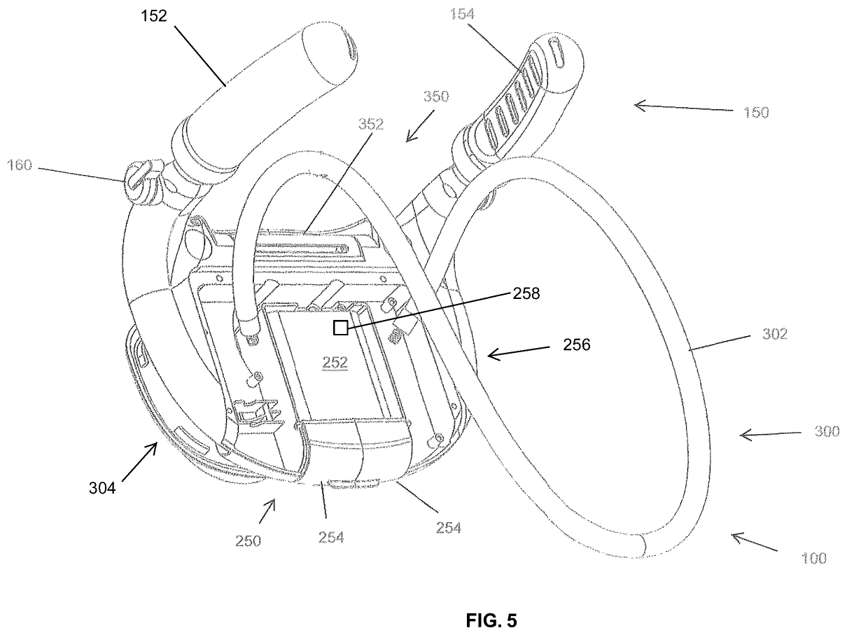

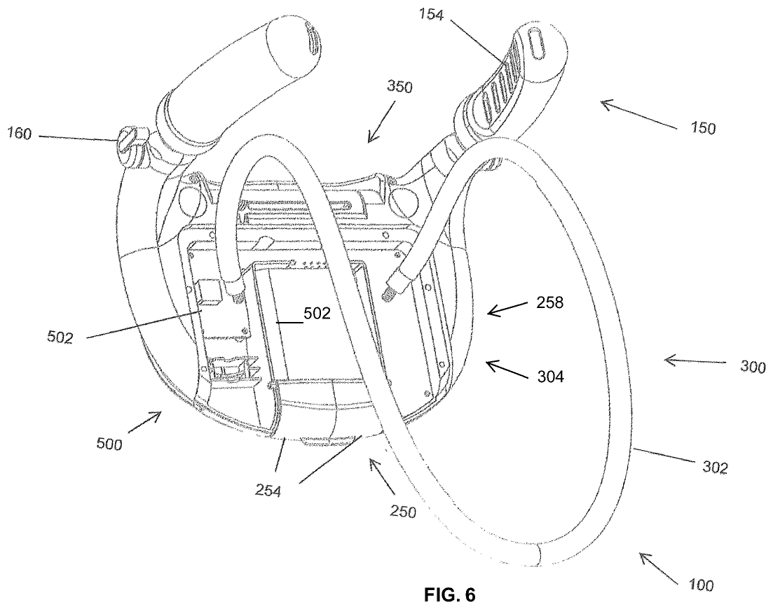

With respect to yoke module 300, as shown in FIGS. 5 and 6, yoke module 300, includes, in part, yoke 302 and yoke connectors 304. Preferably, yoke 302 is constructed of any suitable, durable, high strength, flexible, medical grade material. Preferably, yoke connectors 304 are attached to the back of protective casing 120.

Another unique aspect of the present invention is the use of yoke module 300. In particular, yoke module 300 can be used to assist in retaining ventilation system 100 on the shoulders of the wearer 6. Furthermore, yoke 302 is removably attached to protective casing through the use of yoke connectors 304. In this manner, yoke 302 can be easily attached to and removed from protective casing 120. Furthermore, since yoke 302 is flexible, yoke 302 can be adjusted to fit the upper torso of the wearer 6 so that ventilation system 100 will remain securely retained on the shoulders and the upper torso of the wearer 6. For example, wearer 6 can position the ventilation system 100 with the yoke module 300 installed over his/her head and place the yoke module 300 on the upper torso of the wearer 6 (FIG. 1). The wearer 6 can then pull/push on yoke 302 while yoke 302 is connected to yoke connectors 304 so that yoke 302 firmly contacts the upper torso of the wearer 6 to assist in retaining the ventilation system 100 on the shoulders and upper torso of the wearer 6.

### Neck Vent Module 350

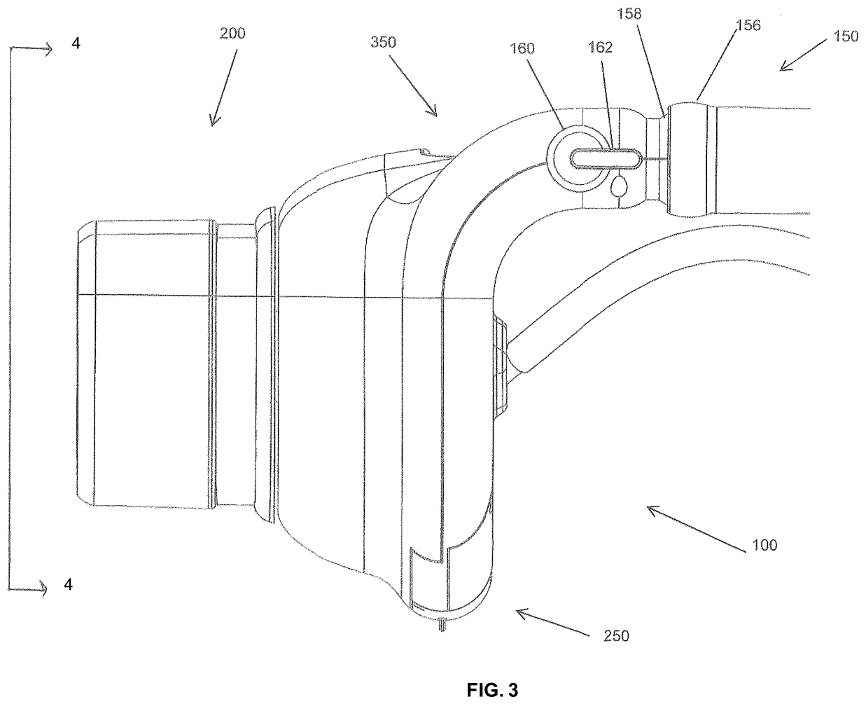

Regarding neck vent module 350, as shown in FIGS. 3-6, neck vent module 350 includes, in part, neck vent 352 (FIG. 4). Preferably, neck vent 352 is constructed of any suitable, durable, high strength, medical grade material.

### Air Flow Generation Module 450

Regarding air flow generation module 450, as shown in FIG. 4, air flow generation module 450 includes, in part, conventional fan motor 260, conventional impeller 454, and back flow opening 456. It is to be understood that battery 252 provides the electrical power to fan motor 260.

Another unique aspect of the present invention is the use of air flow generation module 450. In particular, as the fan motor 260 causes the impeller (or fan) 454 to rotate, the configuration of the impeller 454 causes air to be drawn through the air filter module 200. In this manner, the air filter module 200 can be used to filter the air being drawn into the ventilation system 100. Also, the back flow opening 456 is provided to allow air that is contained within the surgical hood 14 to also be drawn through back flow opening 456 in the direction of arrow D. In this manner, the back flow opening 456 provides for an even greater circulation of the air within the hood 14 while the ventilation system 100 is in operation.

### Printed Circuit Board (PCB) Module 500

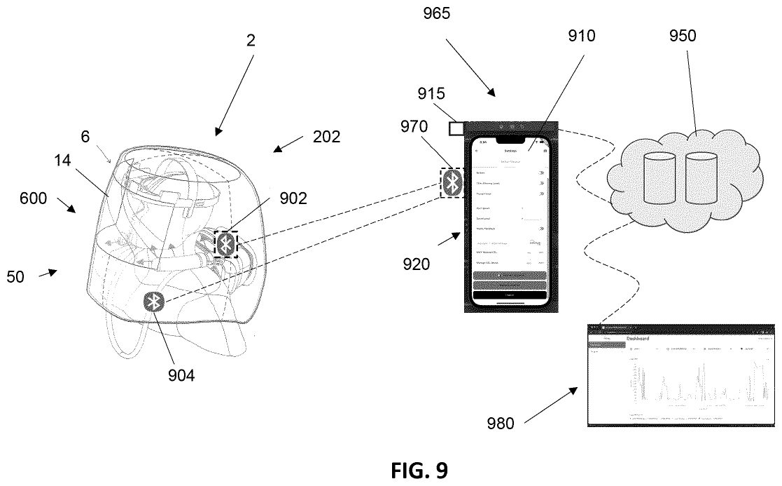

With respect to printed circuit board (PCB) module 500, as shown in FIG. 6, printed circuit board module 500, includes, in part, a conventional printed circuit board 502. It is to be understood that printed circuit board 502 can be used to control the ventilation system 100 and interact with gown wireless identification system 30, air filtration module wireless identification system 208, power module wireless identification system 258, motor sensor/wireless identification system 262, and operating condition parameter measurement assembly 600, as will be described in greater detail later. In particular, printed circuit board 502 can be used to control the speed at which the impeller 454 (FIG. 4) rotates, thereby controlling the velocity of the air being emitted from the face vents 152 and the neck vent 352. It is to be further understood that the printed circuit board 502 is located in the rear of the protective casing 120 so that the printed circuit board 502 can be located adjacent to battery 252. It is to be further understood that the printed circuit board 502 is conventionally retained within the protective casing 102 by conventional fasteners (not shown). Finally, it is to be understood that the printed circuit board (PCB) module 500 can utilize Bluetooth® low energy capabilities in order to allow the printed circuit board (PCB) module 500 to communicate with a mobile application 920 (FIG. 9) that is conventionally installed on a remote computer 965 (i.e., mobile communication device) (FIGS. 9 and 19) such as a smartphone, tablet or data collection point.

### Operating Condition Parameter Measurement Assembly 600

Regarding operating condition parameter measurement assembly 600, as shown in FIGS. 1, 4, and 7, operating condition parameter measurement assembly 600, includes, in part, hood 14, helmetless surgical hood and gown support 50, operating condition parameter measurement assembly 600 which includes an operating condition parameter sensor 602 (and/or 602a in FIG. 4) and a microphone assembly 604. In one embodiment, operating condition parameter sensor 602 and/or 602a can include, but is not limited to, a carbon dioxide (CO₂) sensor, a temperature sensor, a humidity sensor, oxygen (O₂) sensor, volatile organic compounds (VOCs) sensor, and/or an air pressure level sensor. The operating condition parameter sensor 602 (and/or 602a) can be used to measure air quality within hood 14 while the hood 14 is being worn by the user. In one embodiment, the air quality can be related to, but not limited to, a carbon dioxide (CO₂) level, a temperature level, a humidity level, an oxygen (O₂) level, a volatile organic compounds (VOCs) level, and/or an air pressure level within the hood 14 while the hood 14 is being worn by the user.

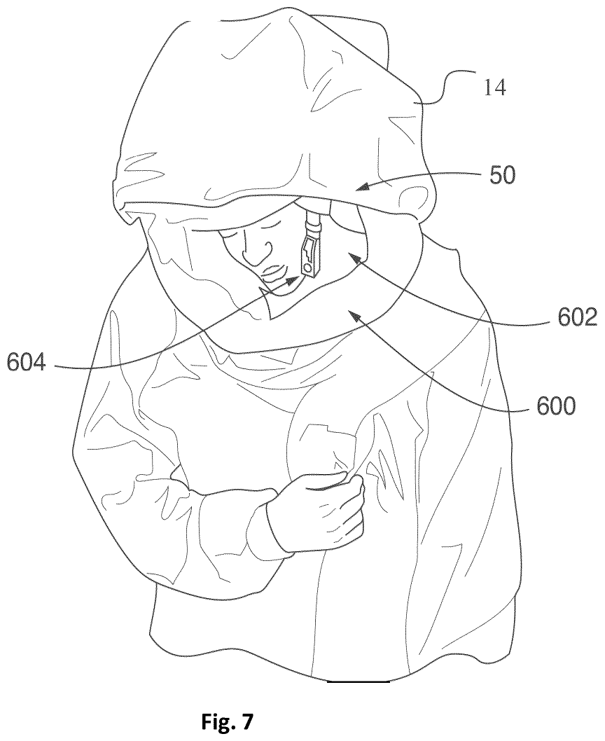

As shown in FIGS. 1 and 7, in one embodiment, the operating condition parameter sensor 602 is connected to the helmetless surgical hood and gown support 50 so that operating condition parameter sensor 602 can be securely located adjacent to the mouth of the wearer. It is to be understood that operating condition parameter measurement assembly 600 can be adjusted so as to be able to position the operating parameter condition measurement assembly 600 within a desired distance away from the wearer's mouth. It is to be further understood that while the operating condition parameter sensor 602 is being used in conjunction with the helmetless surgical hood and gown support 50 and hood 14, operating condition parameter sensor 602 can be used on other medical devices located within the hood 14. Furthermore, operating condition parameter sensor 602 could be used in conjunction with filter 202 to measure the quality of the air that is being introduced into filter 202.

In another embodiment, operating condition parameter measurement assembly 600 can also include an operating condition parameter sensor 602a which can also be located adjacent to the fan motor 250 (FIG. 4) instead of being located adjacent to the user's mouth (FIG. 7). In still another embodiment, both operating condition parameter sensor 602 and 602a can be utilized within hood 14. It is to be understood no matter if either or both operating condition parameter sensor 602 and/or 602a are utilized within hood 14, the operating condition parameter sensor 602 and/or 602a should be located within the hood 14 so that the operating condition parameter sensor 602 and/or 602a can monitor the operating condition parameters or air quality (i.e., CO₂ levels, temperature, humidity levels, oxygen (O₂) levels, volatile organic compounds (VOCs), etc.) within the hood 14 while the hood 14 is being used during a medical procedure.

In another embodiment, the operating condition parameter measurement assembly 600 can also be equipped with a microphone assembly 604. In this manner, the microphone assembly 604 can be used to allow the wearer to communicate with other personnel in the area where the medical procedure is being performed and/or personnel who are observing the medical procedure at a location remote from the medical procedure area and communicate with the mobile communication device 965 associated with the user 6 (FIG. 9), as will be described in greater detail later.

### Operation of System for Managing Medical Device Maintenance and Medical Device Consumables

With respect to the operation of the system for managing medical device maintenance and medical device consumables, attention is directed to FIGS. 1-9. Assume that a medical device such as a surgical gown 4 (FIG. 1) is equipped with a conventional gown wireless identification system 30 having an RFID tag 35 that can be conventionally attached to or electrically connected to the surgical gown 4, as discussed earlier. Secondly, assume that another medical device such as a fan filter 202 (FIG. 4) is equipped with a conventional wireless identification system 208 having an RFID tag 209 that can be conventionally attached to or electronically connected to the air filter 202. Thirdly, assume that a still another medical device such as a battery 252 (FIGS. 4 and 5) is equipped with a conventional power module wireless identification system 258 having an RFID tag 259 that can be conventionally attached to or electronically connected to the battery 252. Fourthly, assume that a fan motor 260 (FIG. 4) is equipped with a motor sensor/wireless identification system 262 having a tachometer 263 and RFID tag 267 that can be conventionally attached to or electronically connected to the fan motor 260. It is to be understood that a processor 2002 (FIG. 19) in conjunction with printed circuit board (PCB) module 500 can use the information from tachometer 263 or any other similar device in the motor sensor/wireless identification system 262 to determine the speed at which the motor 260 is operating and compare that speed to the speed at which the motor 260 should be preferably operating in order to determine if the filter 202 is clogged, the motor 206 is damaged, an operating condition parameter threshold has been exceeded, and/or the system 4 is otherwise malfunctioning to alert the user. Finally, assume that gown wireless identification system 30, air filtration module wireless identification system 208, power module wireless identification system 258, motor sensor/wireless identification system 262, and the operating condition parameter measurement assembly 600 are in electrical communication with printed circuit board 502.

In another embodiment, information from tachometer 263 or any other similar device in the motor sensor/wireless identification system 262 can be sent through the printed circuit board 502 to processor 2002 so that processor 2002 in conjunction with printed circuit board (PCB) module 500 can control the speed of fan motor 260. In particular, operating condition parameter measurement assembly 600 (i.e., operating condition parameter sensor 602 and/or 602a) can be used to provide information to processor 2002 regarding the operating conditions within the hood 14 (FIG. 7). For example, operating condition parameter sensor 602 and/or 602a can be used to detect carbon dioxide (CO₂), temperature, humidity, oxygen (O₂), VOCs, and/or air pressure levels within hood 14. The operating conditions can be monitored while the wearer is donning the hood 14 such as during a medical procedure. The operating conditions data from operating conditions parameter sensor 602 and 602a can be transmitted through the printed circuit board (PCB) module 500 to the processor 2002 at regular intervals such as every thirty (30) seconds during the medical procedure. In another embodiment, the processor 2002 has been configured with operating condition parameter thresholds as set by the user or a system administrator (such as the CO₂ level should not exceed 1,200 ppm or 2,500 ppm depending upon the desired operating conditions), as will be discussed in greater detail later. As discussed above, an algorithm running on the printed control board 265 determines the operating speed of the fan motor 260 based on one or more pre-determined thresholds, and the algorithm could be updated with data from the processor 2002 (FIG. 19) and uploaded onto the printed control board 265 at a desired interval such as every thirty (30) seconds while the medical procedure is being conducted.

During the medical procedure, if at least one of the pre-set thresholds of an operating condition (carbon dioxide (CO₂), temperature, humidity, oxygen (O₂), VOCs, and/or air pressure levels) is exceeded, the processor 2002 in conjunction with printed circuit board (PCB) module 500 can then interact with the fan motor 260 to adjust the speed of the fan motor 260. In one embodiment, the tachometer 263 can then be utilized to monitor the adjustment in the speed of the fan motor 260 so that the desired adjustment in the speed of the fan motor 260 is achieved. For example, the processor 2002 in conjunction with printed circuit board (PCB) module 500 can interact with the fan motor 260 to cause the fan motor 260 to quickly accelerate in speed in order to provide a large amount of fresh air inside of hood 14 if at least one of the thresholds of an operating condition (carbon dioxide (CO₂), temperature, humidity, oxygen (O₂), VOCs, and/or air pressure levels) is exceeded, as discussed earlier. For example, if the CO₂ levels within hood 14 exceed 1,200 ppm (or 2,500 ppm depending upon the operating conditions), the introduction of large amount of fresh air into the hood 14 over a short period of time may correct the operating condition in which the threshold of this operation condition has been exceeded. The operating parameter sensor 602 and/or 602a will continuously monitor the operating conditions within hood 14 while the wearer is donning the hood 14 to determine that no other operating conditions within hood 14 are exceeding their pre-set operating thresholds.

In still another embodiment, the processor 2002 in conjunction with printed circuit board (PCB) module 500 can interact with the fan motor 260 to cause the fan motor 260 to gradually accelerate or decelerate in speed in order to provide a desired amount of fresh air inside of hood 14 if at least one of the pre-set thresholds of an operating condition (carbon dioxide (CO₂), temperature, humidity, oxygen (O₂), VOCs, and/or air pressure levels) is exceeded. For example, if the CO₂ level within hood 14 exceeds a pre-set threshold of 2,500 ppm, the fan motor 260 can be activated to begin to introduce an amount of fresh air into the hood 14 in order to correct the operating condition in which the pre-set threshold of this operation condition has been exceeded. In this example, once it has been determined that the CO₂ levels are below the pre-set threshold of 2,500 ppm, the fan motor 260 can be decelerated or turned off to cause the fan motor 260 to slow or stop the delivery of fresh air into the hood 14. As discussed above, the operating parameter sensor 602 and/or 602a will continuously monitor the operating conditions within hood 14 while the wearer is donning the hood 14 to determine that no other operating conditions within hood 14 are exceeding their pre-set operating thresholds.

In another embodiment, the tachometer 263 can then be utilized to monitor a “full” condition. For example, tachometer 263 can be used to detect if filter 202 is clogged, motor 260 is failing, or the like. In any of these instances, the processor 2002 in conjunction with printed circuit board (PCB) module 500 can then be configured to send an alert to the user informing the user that a “full” condition exists and needs to be looked at.

In another embodiment, the operating condition information forwarded from the operating parameter sensor 602 and/or 602a to the processor 2002 can also be stored in data storage 2006 or forwarded to a remote computer 965 associated with the wearer 6 or a system administrator so that the wearer 6 and/or system administrator will have access to the operating conditions within the hood 14 while the wearer is donning the hood 14. For example, the remote computer 965 may be equipped with a graphical user interface 910 (FIG. 9) that will allow the wearer 6 and/or the system administrator to view the operating conditions within the hood 14 in a graphical format or some other similar format that will allow the wearer 6 and/or system administrator to quickly and easily ascertain if there are any operating conditions within the hood 14 are exceeding the pre-set threshold values while the wearer is donning the hood. Furthermore, the information regarding the operating conditions can be used for subsequent wearer edification, preventative maintenance, and or product improvement.

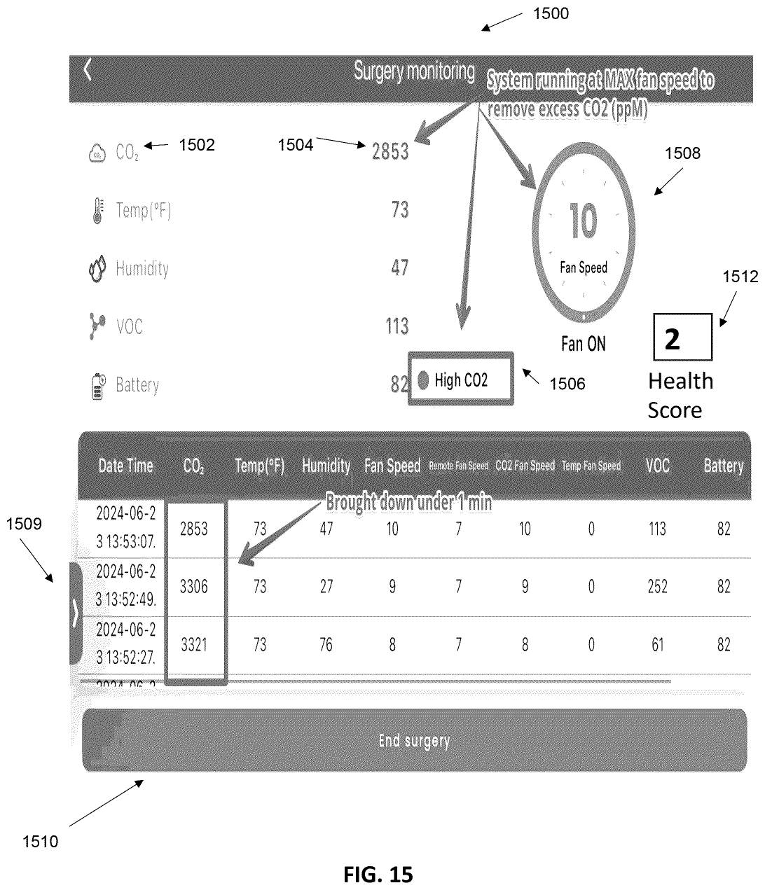

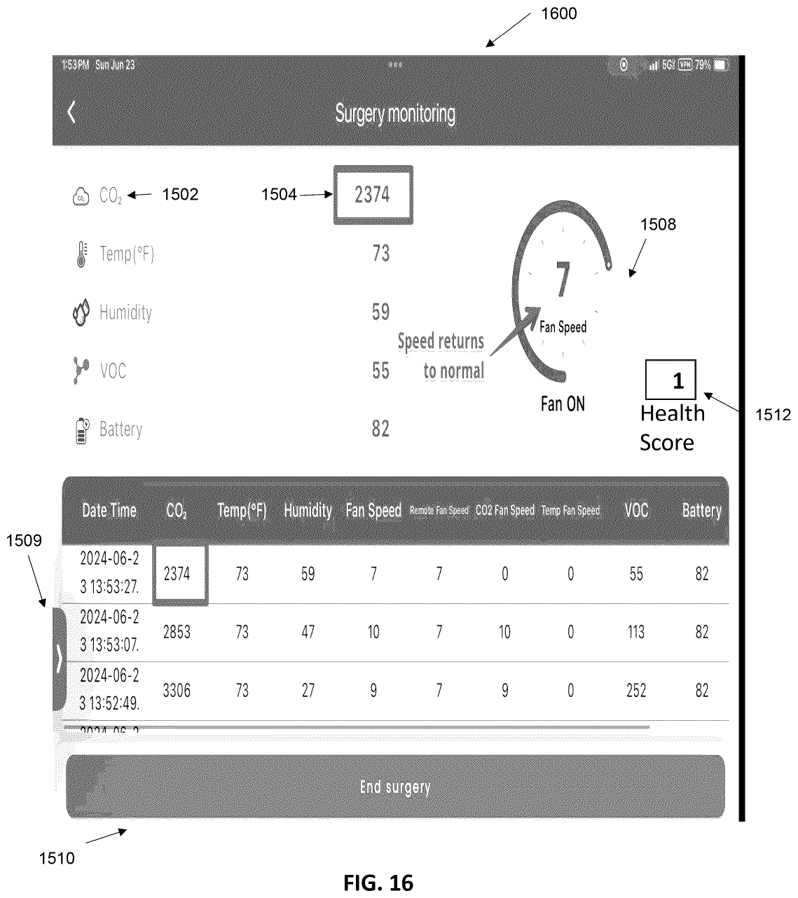

In one embodiment, the wearer 6 and/or system administrator can be provided with an operating parameters (or air quality) health score. In this manner, the wearer and/or system administrator can be provided with a color score such as a green/yellow/red (or number) score. In this embodiment, the color would provide the wearer and/or system administrator with information about the air quality within hood 14 while the wearer 6 is performing the medical procedure. For example, as shown in FIGS. 15 and 16, the color green (or a number such as 1) could be associated with a good health score 1512 (the pre-set thresholds of an operating condition were not exceeded within the hood 14 during the medical procedure). The color yellow (or a number such as 2) could be associated with a fair health score (only 1 or 2 of the pre-set thresholds of an operating condition were exceeded within the hood 14 during the medical procedure). The color red (or a number such as 3) could be associated with a poor health score (many of the pre-set thresholds of an operating condition were exceeded within the hood 14 during the medical procedure).

Furthermore, operating conditions could be monitored every 30 seconds (or other desired period of time). In this manner, if the wearer 6 was performing a medical procedure that required more than usual manual exertion, it is possible that the wearer's CO₂ output, humidity, oxygen (O₂), VOCs, and/or temperature within the hood 14 might be greater than during a less strenuous medical procedure. The information related to the amount of CO₂ that the wearer is exerting, along with the increased humidity, temperature, oxygen (O₂), and VOC levels, could be useful to the wearer 6 and/or the system administrator so that possibly the speed of the fan motor 260 could be automatically increased in similar medical procedures in the future thereby maintaining the CO₂, humidity, oxygen (O₂), VOC, and/or temperature levels within the hood 14 below the threshold levels.

Another unique aspect of the present invention is that the information obtained for use in determining the operating parameter health score can also be used to create best practices for air quality, threshold, and usage settings. In particular, data that is related to the operating parameters (or air quality) health score data and is temporarily stored in memory 2004 under data 2016 can be executed by processor 2002 from a server 950 (FIG. 9) to determine best practices such as fan speed, optimum CO₂ levels, humidity levels, temperature levels, oxygen (O₂) levels, and/or VOC levels. This data can then be used in conjunction with machine learning techniques and artificial intelligence (AI) algorithms to recommend best practices for consumable operation and management in order to improve air quality within the hood 14 and when using the ventilation system 100.

In still another embodiment, processor 2002 can also be configured to send a signal to the remote computer 965 which is configured with an alarm 915 (FIG. 9) such as an audible and haptic (vibrate) alarm to provide an alert to the user 6 to inform the user that at least one of the pre-set thresholds of an operating condition (carbon dioxide (CO₂), temperature, humidity, oxygen (O₂), VOCs, and/or air pressure levels) has been exceeded. In this instance, if the processor 2002 interacts with fan motor 260 upon determining that a pre-set operating condition threshold has been exceeded, but the fan motor speed is not adjusted to attempt to correct the operating condition that is being exceeded, the alarm 915 can provide a backup to the wearer 6 and/or the system administrator that the system 2 needs to be looked at to determine why the system 2 allowed the operating condition to exceed its pre-set operating threshold.

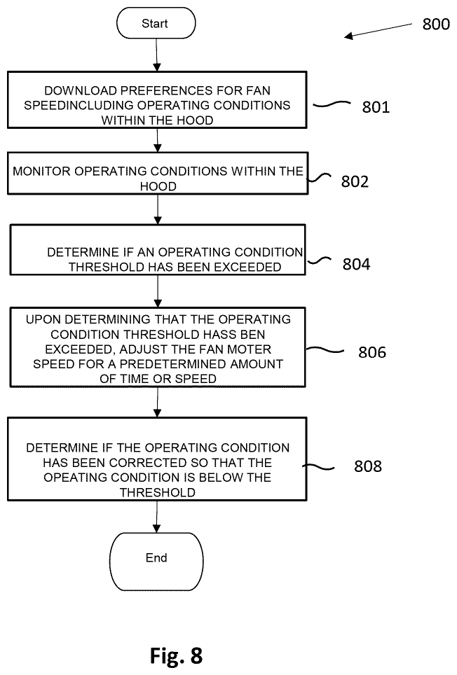

With reference to FIG. 8, a computer-implemented method 800 is illustrated that describes one embodiment for managing operating condition parameters in medical devices. The method 800 is performed by at least a processor 2002 (FIG. 19) of a computer system 2000 that accesses and interacts with memories and/or data storage devices. For example, the processor 2002 at least accesses and reads/writes data to the memory and processes network communications to perform the actions of FIG. 8.

With reference to FIG. 8, at 801, the method 800 is initiated when the end user (usually the wearer 6 of the hood 14 and gown 4) interacts with a remote computer 965 such as a mobile communication device 965 that is configured with a mobile app 920 (iOS and Android) that is “paired with” or otherwise interacts with the medical device (e.g., the hood 14 and/or the ventilation system 100 prior to the medical procedure). In one embodiment, the medical device can be selected by the end user either by selecting from a list of medical devices or scanning the barcode on the medical device before the surgery.

It is to be understood that typically the user 6 would conventionally set up an account with a web site that administers the mobile app 920 through the mobile communication device 965. In particular, the user 6 would access the web site and then complete a conventional registration process that includes setting up a user name and a password that would allow the user 6 to access the user's information on the mobile app 920.

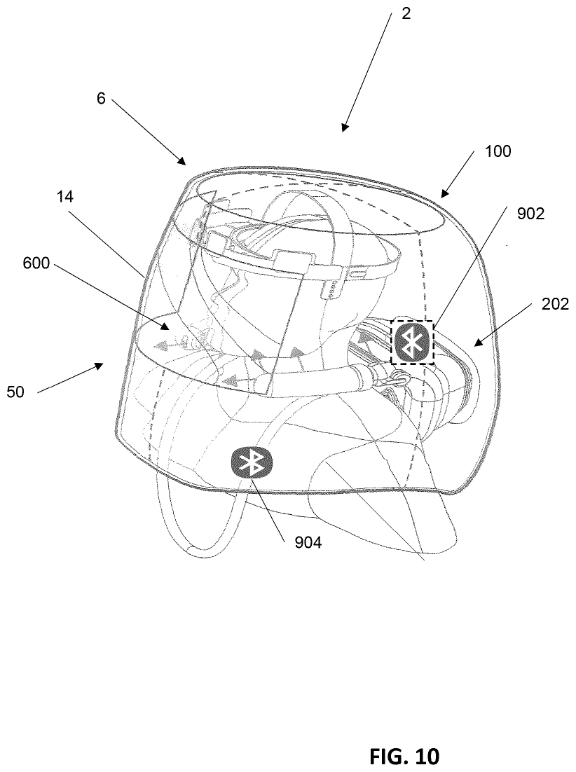

With respect to 801, attention is directed to FIGS. 9-12. In particular, as shown in FIGS. 9 and 10, as discussed above, in one embodiment, the system 2 includes, in part, user 6, hood 14, helmetless surgical hood and gown support 50, air filter 202, operating parameter measurement assembly 600, transceiver 902, wireless remote control 904, remote computer 965 which is configured with a display 910, an alarm 915, and a transceiver 970, cloud storage 950, and progressive web application (PWA) 980.

A unique aspect of the present invention is the use of the helmetless surgical hood and gown support 50 and air filter 202 in conjunction with the operating parameter measurement assembly 600, transceiver 902, wireless remote control 904. In particular, during a medical procedure, data related to the operating conditions within the hood 14 can be taken at regular intervals such as every thirty (30) seconds using the operating parameter measurement assembly 600. This data can be related to the CO₂, humidity, oxygen (O₂), VOC, and/or temperature levels within the hood 14. The data is sent from the operating parameter measurement assembly 600 to the processor 2002. The processor 2002 is configured to then transfer the data from the processor 2002 to the remote computer 965 through the transceivers 902 and 970.

With respect to information regarding the operation of the battery 252, the battery sensor 253 can be used to monitor a state of the battery charge (in percentage), a battery cycle count, and/or battery wear. This information can then be transferred from the battery sensor 253 (FIG. 4) to the processor 2002. The processor 2002 is configured to then transfer the data from the processor 2002 to the remote computer 965 through the transceivers 902 and 970.

It is to be further understood that information regarding the operation the fan motor 260 (FIG. 4), the tachometer 263 can be used to provide information on the operating speed of the fan motor 260. Also, the information can be related to the speed at which the fan motor 260 has been originally set and the actual speed of the fan motor 260 can be stored on the printed circuit board 265. This fan motor speed information can be forwarded from the tachometer 263 to the printed circuit board 265 and then to the processor 2002. The processor 2002 is configured to then transfer the data from the processor 2002 to the remote computer 965 through the transceivers 902 and 970.

It is to be further understood that information regarding the consumables such as the surgical gown 4 and the air filter 202 can be collected. For example, the RFID tag 35 that is conventionally attached to the surgical gown 4 can be used to provide information on the surgical gown 4 such as when the expiration date of the surgical gown 4, the surgical gown serial number, the manufacturer of the surgical gown 4, or the like. In a similar manner, the RFID tag 209 that is conventionally attached to the air filter 202 can be used to provide information on the air filter 202 such as when the expiration date of the air filter 202, the air filter serial number, the manufacturer of the air filter 202, or the like.

Another unique aspect of the present invention is the use of wireless remote control 904. In one embodiment, the wireless remote control 904 is configured with Bluetooth® capability to be able to interact with the remote computer 965 in order to control the system 2.

A still another unique aspect of the present invention is the use of remote computer 265 which is configured with a display 910, an alarm 915, a mobile application 920 that is conventionally installed on the remote computer 265, and a transceiver 970. In particular, in one embodiment, the remote computer 265 in conjunction with the display 910, the mobile application 920, and the transceiver 970 can be used to control the system 2.

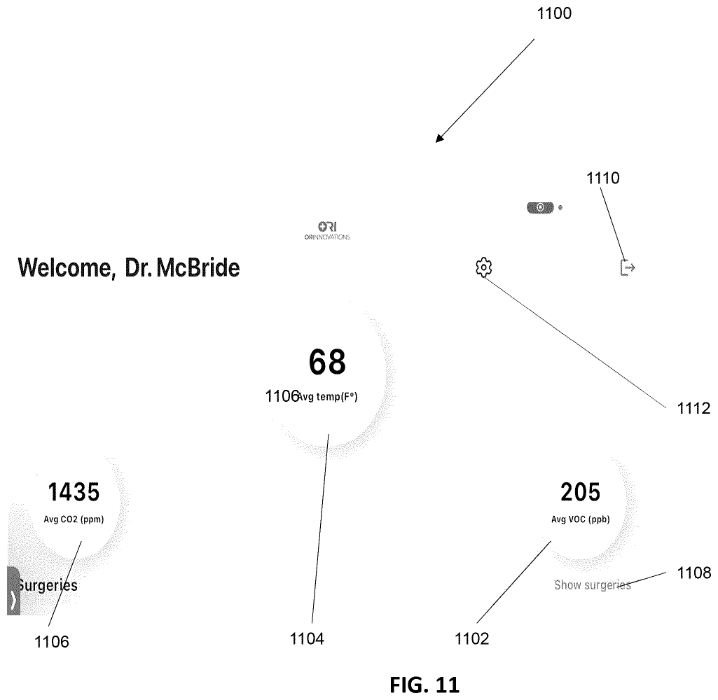

Attention is now directed to FIGS. 9 and 11, with respect to step 801, as discussed above, wearer 6 of the hood 14 and gown 4 interacts with a remote computer 965 that is configured with the mobile app 920 (iOS and Android) that is “paired with” or otherwise interacts with the hood 14 and/or the ventilation system 100 prior to the medical procedure. Upon interacting with the remote computer 965, the remote computer 265 is configured so that a login and welcome home page 1100 (FIG. 11) is caused to be displayed on display 910.

As shown in FIG. 11, in one embodiment, while the hood 14 is being worn during a medical procedure, login and welcome home page 1100 is configured to display an average VOC level 1102 within the hood, an average temperature level 1104 within the hood 14, and an average CO₂ level 1106 within the hood. It is to be understood that the average VOC level, temperature level, and the CO₂ level can be obtained from the operating parameter measurement assembly 600, as discussed earlier. This information can then be transferred to the processor 2002 and then to the mobile app 920 thorough the transceivers 902 and 970, as discussed earlier. It is to be understood that the mobile app 920 can be configured to be able to conventionally determine the average VOC level, temperature level, and the CO₂ level and then display these average levels.

A unique aspect of the present invention is the displaying of the average VOC level, temperature level, and the CO₂ level. In particular, the average VOC level, temperature level, and the CO₂ level can used reviewed by the user 6 or a system administrator to determine if the user 6 is being subjected to any harmful conditions within the hood while the user 6 is donning the hood. For example, if it is known that the VOC content within a hood should not exceed 205 ppb, the system 2 can then be adjusted so that the user does not encounter average VOC levels that exceed 205 ppb, as will be discussed in greater detail later.

As shown in FIG. 11, the login and welcome home page 1100 is configured to include a next page button 1110. This will allow the user 6 to scroll to the next page of the mobile app 920.

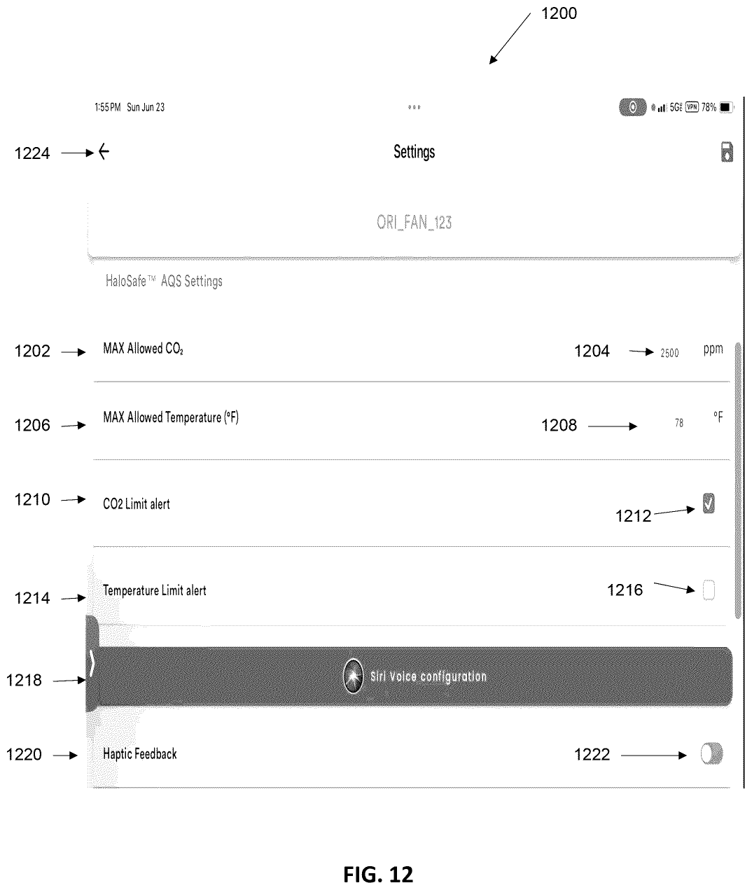

As shown in FIG. 11, the login and welcome home page 1100 is configured to include a setting page button 1112. This will allow the user 6 to move to the settings page 1200 (FIG. 12) of the mobile app 920.

Finally, as shown in FIG. 11, login and welcome home page 1100 is configured to include a show surgeries button 1108 that allows the user to see the surgeries that the user completed.

As shown in FIG. 12, there is illustrated a settings page 1200. In one embodiment, the settings page 1200 is configured to display, in part, a maximum allowed CO₂ setting 1202 that allows the user 6 to select a desired allowed CO₂ level (in ppm) 1204, a maximum allowed temperature setting 1206 that allows the user 6 to select a desired allowed temperature (in ° F) 1208, a CO₂ limit setting 1210 having a check box 1212 that allows the user to select if the user 6 wants to receive a CO₂ alert through the remote computer 965, a temperature limit alert 1214 having a check box 1216 that allows the user to select if the user 6 wants to receive a temperature alert through the remote computer 965, a virtual assistant voice recognition setting 1218 that allows the user 6 to utilize the microphone assembly 604 in conjunction with the remote computer 965 and the mobile app 920 to control the system 2, and a haptic (vibration) feedback setting 1220 having a toggle button 1222 that allows the user to select if the user 6 wants to receive a haptic feedback (vibrations) through the remote computer 965.

It is to be understood that while CO₂ and temperature settings are shown in FIG. 12, settings for other operational information such as humidity, oxygen (O₂), volatile organic compounds (VOCs), and/or air pressure within the surgical hood 14 while the surgical hood 14 is being worn can also be included in the settings page 1200. Also, settings for battery low alert, clogged air filter 202 alert, battery replace alert, and/or air filter replace alert can also be included in the settings page 1200.

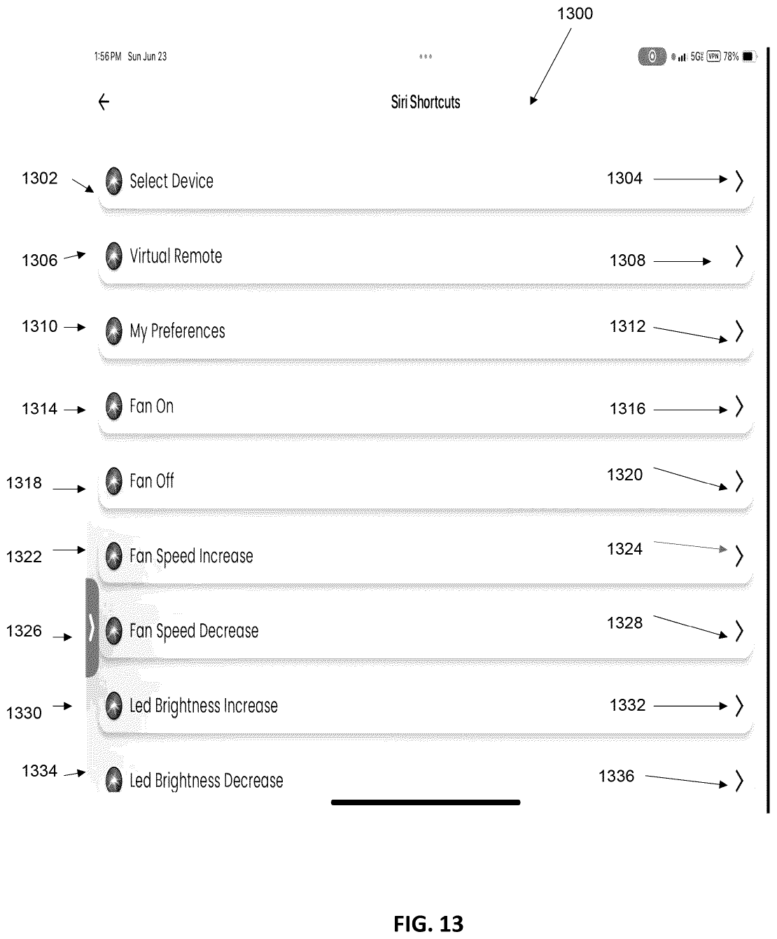

Another unique aspect of the present invention is the use of the microphone assembly 604 in conjunction with the remote computer 965 and the mobile app 920 to control the system 2. For example, the user 6 may interact with remote computer 965 that has been configured with a virtual assistant such as Siri, Alexa, or Google Assistant. The user 6 can then utilize the virtual assistant to set up various settings for operating system 2. For example, as shown in FIG. 13, in one embodiment, the mobile app 920 can be configured to display a virtual assistant settings page 1300 that will allow the user 6 to control various operating conditions of the system 2. In particular, the virtual assistant settings page 1300 can include a select a medical device setting 1302 that includes a selection button 1034. The user 6 can then select button 1304 in order to select a desired medical device such as hood 14. The virtual assistant settings page 1300 can include a virtual remote setting 1306 that includes a select button 1308 that allows the user 6 to select whether the user 6 wants to use a virtual remote. The virtual assistant settings page 1300 can include a my preferences setting 1310 that includes a select button 1312 that allows the virtual assistant to provide the user 6 with a list of the user's preferences, as discussed above with reference to FIG. 12. The virtual assistant settings page 1300 can include a fan on setting 1314 that includes a select button 1316 that allows the user 6 to turn on the fan motor 260. The virtual assistant settings page 1300 can include a fan off setting 1318 that includes a select button 1320 that allows the user 6 to turn off the fan motor 260. The virtual assistant settings page 1300 can include a fan speed increase setting 1322 that includes a select button 1324 that allows the user 6 to increase the speed of the fan motor 260. The virtual assistant settings page 1300 can include a fan speed decrease setting 1326 that includes a select button 1328 that allows the user 6 to decrease the speed of the fan motor 260. The virtual assistant settings page 1300 can include a light brightness increase setting 1330 that includes a select button 1332 that allows the user 6 to increase the brightness of a light located within the hood 14 (not shown). The virtual assistant settings page 1300 can include a light brightness decrease setting 1334 that includes a select button 1336 that allows the user 6 to decrease the brightness of a light located within the hood 14 (not shown).

It is to be understood that the end user can either download the end user's system 2 preferences through the remote computer 965 to the processor 2002 where the preferences are stored in the data storage 2006. In another embodiment, the user 6 can also download and store threshold values for the operating conditions such as, but not limited to, carbon dioxide (CO₂), temperature, humidity, oxygen (O₂), VOC, and/or air pressure levels.

With reference to FIG. 8, at 802, the method 800 is initiated when operating condition parameter sensor 602 and/or 602a monitor the operating conditions within hood 14 (FIG. 7) while the wearer is donning the hood 14. As discussed above, the operating condition parameter sensor 602 and/or 602a can be used to monitor operating parameter conditions such as, but not limited to, carbon dioxide (CO₂), temperature, humidity, oxygen (O₂), VOCs, and/or air pressure levels.

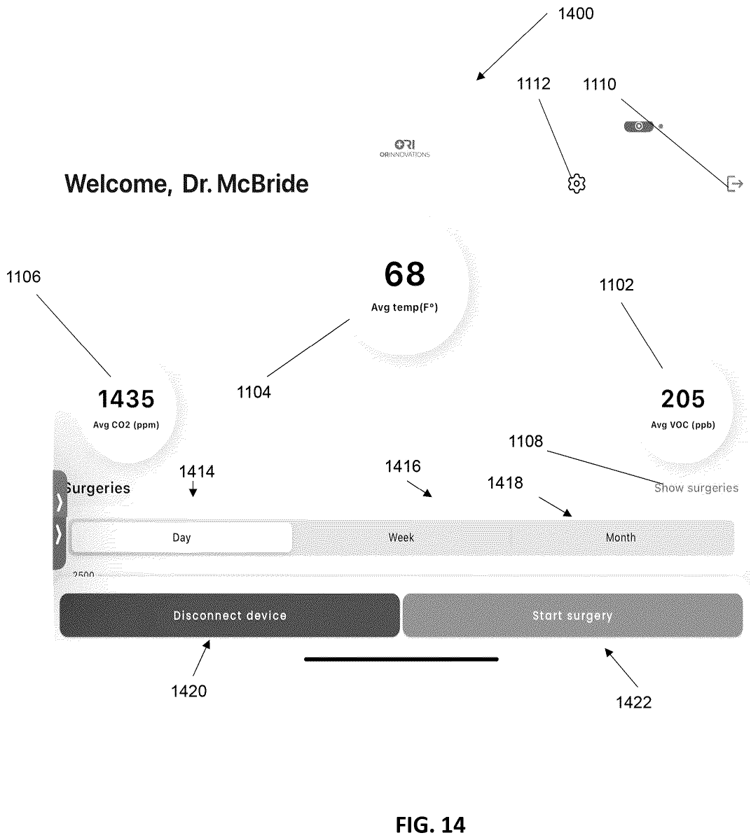

In particular, as shown in FIG. 14, a system operation page 1400 is configured to display an average VOC level 1102 within the hood 14, an average temperature level 1104 within the hood 14, and an average CO₂ level 1106 within the hood. It is to be understood that the average VOC level, temperature level, and the CO₂ level can be obtained from the operating parameter measurement assembly 600, as discussed earlier. This information can then be transferred to the processor 2002 to the mobile app 920 thorough the transceivers 902 and 970, as discussed earlier. It is to be understood that the mobile app 920 can be configured to be able to conventionally determine the average VOC level, temperature level, and the CO₂ level and then display these average levels.

As shown in FIG. 14, the system operation page 1400 is configured to include a next page button 1110. This will allow the user 6 to scroll to the next page of the mobile app 920.

As shown in FIG. 14, the system operation page 1400 is configured to include a setting page button 1112. This will allow the user 6 to move to the settings page 1200 (FIG. 12) of the mobile app 920.

As shown in FIG. 14, the system operation page 1400 is configured with day 1414, week 1416, and month 1418 buttons that allow the user to view an average VOC level 1102 within the hood 14, an average temperature level 1104 within the hood 14, and an average CO₂ level 1106 within the hood 14 over a particular day, week or month.

As shown in FIG. 14, the system operation page 1400 is configured with a disconnect device button 1420 that allows the user 6 to disconnect the mobile device 965 from the mobile app 920 when, for example, the medical procedure is completed. Furthermore, the system operation page 1400 is configured with a start surgery button 1422 that allows the user 6 to connect the mobile device 965 to the mobile app 920 when, for example, the medical procedure has begun. In this instance, the mobile device 965 can begin transferring information from the operating parameter measurement assembly 600 to the mobile device 965 and then to the mobile app 920.

Returning to method 800, in block 804, in one embodiment, if at least one operating condition parameter has exceeded an operating parameter threshold (e.g., the CO₂ level within the hood 14 has exceeded 1,200 ppm or 2,500 ppm depending upon the operating conditions), the operating condition parameter sensor 602 and/or 602a will determine this high level of CO₂ within hood 14 and forward this information to processor 2002 which, in turn, transfers the information to the mobile computer 965 and the mobile app 920.

In particular, as shown in FIG. 15, assume that at some point during the medical procedure while the user 6 was wearing the hood 14, the CO₂ level within the hood 14 has exceeded 2,500 ppm. Also, assume that the user 6 previously set a CO₂ level limit of 2,500 ppm (1202 and 1204) and a CO₂ limit alert 1210, as discussed above with reference to FIG. 12. As discussed above, the mobile app 920 is configured to then send an alert to the user through the mobile device 965 which is configured to display the alert page 1500. As shown in FIG. 15, the alert page 1500 is configured to be able to display to the user 6 the current CO₂ level of 2853 ppm which exceeded the user pre-set preference CO₂ limit of 2,500 ppm (1502 and 1504). The mobile app 920 is also configured to provide the user 6 with an alert 1506 that informs the user of a high CO₂ level. Finally, since the pre-set preference of CO₂ limit level of 2,500 ppm, the system 2 is configured to turn up the speed of the fam motor 260 to a high level (i.e., 10) so that the system 2 can quicky reduce the CO₂ level within the hood 14, as discussed earlier. As shown in FIG. 15, the alert page 1500 is configured to be able to display to the user 6 the current fan speed (i.e., 10) 1508.

Another unique aspect of the present invention is the display of the various operating conditions of the system 2 over a period of time during the medical procedure. As shown in FIG. 15, mobile app 920 is configured to display the operating condition parameter levels 1509 within the hood 14 over a specified interval during the medical procedure (i.e., every 20-20 seconds). In this manner, the user 6 (or a system administrator) can monitor the system 2 and the operating conditions within the hood 14 while the user 6 is wearing the hood 14 during a medical procedure. This allows the user 6 and/or the system administrator to review the operating parameter conditions of the medical devices for user edification and/or product improvement.

Finally, the alert page 1500 is configured with an end surgery button 1510. The end surgery button 1510 is configured to allow the user 6 to interact with the end surgery button 1510 to stop the display of the alert page 1500 when the surgical procedure has been completed.

In block 806, upon determining that at least one operating condition parameter has exceeded its operating condition threshold, the processor 2002 in conjunction with printed circuit board (PCB) module 500 can then interact with fan motor 260 in order to adjust the speed of the fan motor 260.

In one embodiment, the fan motor 260 can be adjusted to operate the impeller 454 at a “constant” setting. For example, the wearer may set an initial fan speed at 2 out of 10. If the CO₂ operating parameter condition within hood 14 is detected above 1,200 ppm (or 2,500 ppm depending upon the operating conditions), the processor 2002 can also be configured to send a signal to an alarm 915 (FIG. 9) such as an audible alarm (such as a beep) to the wearer and/or a haptic (vibrate) alarm (such as a vibration from the fan motor 260) to provide an alert to the user to inform the user that at least one of the thresholds of an operating condition (carbon dioxide (CO₂), temperature, humidity, oxygen (O₂), VOCs, and/or air pressure levels) has been exceeded. As discussed above, the fan motor 260 can be adjusted to operate the impeller 454 at a desired speed and/or a desired time in order to attempt to correct the operating condition so that the operating parameter condition does not exceed its operating parameter threshold. The processor 2002 in conjunction with printed circuit board (PCB) module 500 will then instruct the fan motor 260 to slowly increase the fan speed to 3, 4, or 5 (which is measured by the tachometer 263) until the CO₂ level as measured by the operating condition parameter sensor 602 and/or 602a goes below the operating condition parameter threshold. Finally, the fan speed will stay at this higher level unless manually turned down by the wearer.

In another embodiment, assume that the wearer has set the operating condition parameter for CO₂ to be at 1,200 ppm and the correction or mitigation method is set to “purge”. The wearer also sets the fan speed to 2 out of 10. If the CO₂ operating condition within hood 14 is detected above 1,200 ppm, the ventilation system 100 may be equipped to provide an alarm (such as a beep) to the wearer and the fan motor 260 may provide an alarm 915 (such as a vibration), as discussed above. The processor 1502 in conjunction with printed circuit board (PCB) module 500 will then instruct the fan motor 260 to quickly increase the fan speed to 9 or 10 (which is measured by the tachometer 263) for a short period until the CO₂ level as measured by the operating condition parameter sensor 602 and/or 602a goes below the operating condition parameter threshold. Finally, the fan speed will return to the initially selected level.

In block 808, the operating condition parameter sensor 602 and/or 602a can be used to monitor the operating parameter conditions within the hood 14 to determine if the operating parameter condition has been corrected so that the operating parameter condition is now below its operating condition parameter threshold.

In particular, as shown in FIG. 16, once the operating parameter condition has been corrected (i.e., the CO₂ level within the hood 14 has been reduced to below 2,500 ppm) while the user 6 was wearing the hood 14, the mobile app 920 is configured to then send an alert to the user through the mobile device 965 which is configured to display the return to normal operating conditions alert page 1600 (such a return to a desired CO₂ level within the hood 14). As shown in FIG. 16, the alert page 1600 is configured to be able to display to the user 6 the current CO₂ level of 2374 ppm which is below the user pre-set preference CO₂ limit of 2,500 ppm (1502 and 1504). Since the pre-set preference of CO₂ limit level is 2,500 ppm, the system 2 is configured to turn down the speed of the fam motor 260 from a high level (i.e., 10) to a lower level (i.e., 7) so that the system 2 can resume an operating speed of the fan motor 260 (i.e., 7) that the user 6 set in the settings page 1200, as discussed earlier.

Another unique aspect of the present invention is the display of the various operating conditions of the system 2 over a period of time during the medical procedure. As shown in FIG. 16, mobile app 920 is configured to display the operating condition parameter levels 1508 within the system 2 over a specified interval during a medical procedure (i.e., every 20-30 seconds). In this manner, the user 6 (or a system administrator) can monitor the system 2 and the operating conditions within the hood 14 on the display 910 of the mobile computer 95 while the user 6 is wearing the hood 14 during a medical procedure. This allows the user 6 and/or the system administrator to review the operating parameter conditions of the medical devices for user edification and/or product improvement.

Finally, the alert page 1600 is configured with an end surgery button 1510. The end surgery button 1510 is configured to allow the user 6 to interact with the end surgery button 1510 to stop the display of the alert page 1500 when the surgical procedure has been completed.

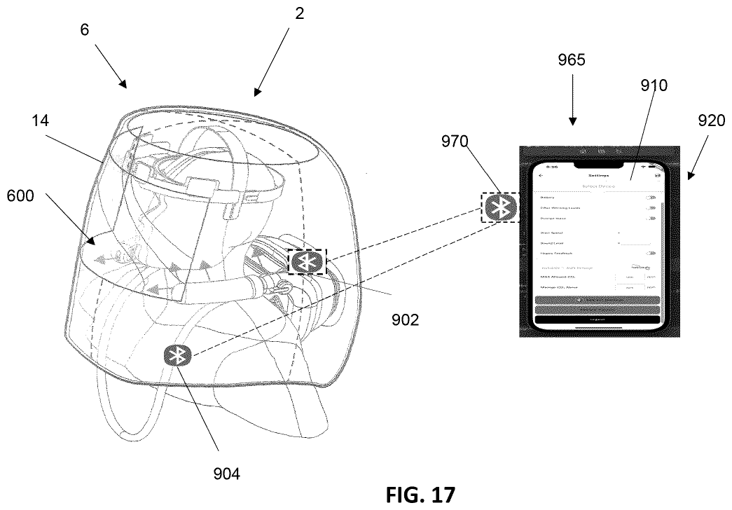

Another unique aspect of the present invention is the interaction between the medical device such as surgical hood 14. As shown in FIG. 17, during a surgical procedure, the operating parameter measurement assembly 600 is collecting information regarding the operating condition parameters within the hood 14 such as the levels of carbon dioxide (CO₂), temperature, humidity, oxygen (O₂), volatile organic compounds (VOCs), and/or air pressure within the surgical hood 14 while the surgical hood 14 is being worn. The information from the operating parameter measurement assembly 600 is forwarded to the processor 2002 and then to the mobile app 920 through the mobile computer 965 through transceivers 902 and 970, as discussed above. The mobile app 920 is configured to compare the current operating condition levels within the hood 14 with the pre-set operating condition parameters that were set by the user 6, as discussed earlier. The mobile app 920 is then configured to send alerts through alert pages 1500 or 1600 to inform the user that either an operating condition has exceeded its limit by 90%, for example, (FIG. 15) or the operating condition level is within a threshold (i.e., 90% of the pre-set limit) as shown in FIG. 16.

It is to be understood that after a period of time (i.e., 30 seconds), the mobile app 920 is configured to display the system operation page 1400. If there is another instance of an operating condition exceeding its pre-set limit, then the mobile app 920 will then cause the mobile computer 965 to display the alert page 1500 and then the alert page 1600, once the condition is remedied, as discussed above.

At the end of the surgical procedure, the operating condition parameter data that was collected by the operating parameter measurement assembly 600 and the system 2 (such as battery and/or air filter data) can be stored in the database 2006 and/or sent to cloud storage 950 (FIG. 9). As discussed above, the stored data can be used for user edification (i.e., surgeon history and/or personal safety) and/or product improvement (replacement best practices for the battery, gown, air filter, or the like).

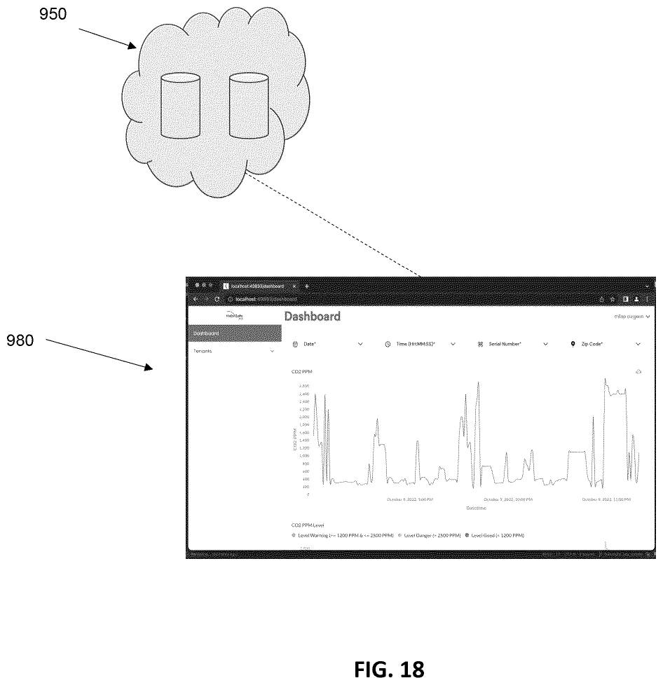

Another unique aspect of the present invention is the interaction between the cloud storage 950 and the progressive web application (PWA) 980. As shown in FIG. 18, since system 2 is configured to allow multiple users 6 to interact with the mobile app 920 through a mobile device 965 associated with each of the user 6, this multi-tenant architecture allows each user 6 (i.e., doctor and/or surgeon) to set up their own accounts or medical facility (i.e., hospital or surgical facility) to set up their own users. Consequently, since the data of the operating condition parameters within the hood 14 that is being worn by a user during a medical procedure is stored in the cloud storage 950, the system 2 can be configured to allow the data for each of the users to be transferred to a conventional PWA 980. The PWA 980 can then be configured for the following:
1. Detect faults within the system 2
2. Proactively recommend best practices for replacement and maintenance of the battery, the filters, the surgical gowns, the motors, and/or other consumables
3. Track life cycle of the system 2, including hours, history, or the like
4. Provide an analysis of the data through the use of graphical presentations.

### Computing Device Embodiment

FIG. 19 illustrates an example computing device that is configured and/or programmed as a special purpose computing device with one or more of the example systems and methods described herein, and/or equivalents. The example computing device may be a computer 900 that includes at least one hardware processor 2002, a memory 2004, and input/output ports 2010 operably connected by a bus 2008. In one example, the computer 2000 may include logic 2030 similar to logic/system 800 shown in FIG. 8.

In different examples, the logic 2030 may be implemented in hardware, a non-transitory computer-readable medium 2037 with stored instructions, firmware, and/or combinations thereof. While the logic 2030 is illustrated as a hardware component attached to the bus 2008, it is to be appreciated that in other embodiments, the logic 2030 could be implemented in the processor 2002, stored in memory 2004, or stored in disk 2006.

In one embodiment, logic 2030 or the computer is a means (e.g., structure: hardware, non-transitory computer-readable medium, firmware) for performing the actions described. In some embodiments, the computing device may be a server operating in a cloud computing system, a server configured in a Software as a Service (SaaS) architecture, a smart phone, laptop, tablet computing device, and so on.

The means may be implemented, for example, as an ASIC programmed to predict a product demand. The means may also be implemented as stored computer executable instructions that are presented to computer 2000 as data 2016 that are temporarily stored in memory 2004 and then executed by processor 2002.

Logic 2030 may also provide means (e.g., hardware, non-transitory computer-readable medium that stores executable instructions, firmware) for storing and measuring operating condition parameters.

Generally describing an example configuration of the computer 2000, the processor 2002 may be a variety of various processors including dual microprocessor and other multi-processor architectures. A memory 2004 may include volatile memory and/or non-volatile memory. Non-volatile memory may include, for example, ROM, PROM, and so on. Volatile memory may include, for example, RAM, SRAM, DRAM, and so on.

A storage disk 2006 may be operably connected to the computer 2000 via, for example, an input/output (I/O) interface (e.g., card, device) 2018 and an input/output port 2010 that are controlled by at least an input/output (I/O) controller 2040. The disk 2006 may be, for example, a magnetic disk drive, a solid-state disk drive, a floppy disk drive, a tape drive, a Zip drive, a flash memory card, a memory stick, and so on. Furthermore, the disk 2006 may be a CD-ROM drive, a CD-R drive, a CD-RW drive, a DVD ROM, and so on. The memory 2004 can store a process 2014 and/or a data 2016, for example. The disk 2006 and/or the memory 2004 can store an operating system that controls and allocates resources of the computer 2000.

The computer 2000 may interact with, control, and/or be controlled by input/output (I/O) devices via the input/output (I/O) controller 2040, the I/O interfaces 2018, and the input/output ports 2010. Input/output devices may include, for example, one or more displays 2070, printers 2072 (such as inkjet, laser, or 3D printers), audio output devices 2074 (such as speakers or headphones), text input devices 2080 (such as keyboards), cursor control devices 2082 for pointing and selection inputs (such as mice, trackballs, touch screens, joysticks, pointing sticks, electronic styluses, electronic pen tablets), audio input devices 2084 (such as microphones or external audio players), video input devices 2086 (such as video and still cameras, or external video players), image scanners 2088, video cards (not shown), disks 2006, network devices 2020, and so on. The input/output ports 2010 may include, for example, serial ports, parallel ports, and USB ports.

The computer 2000 can operate in a network environment and thus may be connected to the network devices 2020 via the I/O interfaces 2018, and/or the I/O ports 2010. Through the network devices 2020, the computer 2000 may interact with a network 2060. Through the network, the computer 2000 may be logically connected to remote computers 965. Networks with which the computer 2000 may interact include, but are not limited to, a LAN, a WAN, and other networks.

### Definitions and Other Embodiments

In another embodiment, the described methods and/or their equivalents may be implemented with computer executable instructions. Thus, in one embodiment, a non-transitory computer readable/storage medium is configured with stored computer executable instructions of an algorithm/executable application that when executed by a machine(s) cause the machine(s) (and/or associated components) to perform the method. Example machines include but are not limited to a processor, a computer, a server operating in a cloud computing system, a server configured in a Software as a Service (Saas) architecture, a smart phone, and so on). In one embodiment, a computing device is implemented with one or more executable algorithms that are configured to perform any of the disclosed methods.

In one or more embodiments, the disclosed methods or their equivalents are performed by either: computer hardware configured to perform the method; or computer instructions embodied in a module stored in a non-transitory computer-readable medium where the instructions are configured as an executable algorithm configured to perform the method when executed by at least a processor of a computing device.

While for purposes of simplicity of explanation, the illustrated methodologies in the figures are shown and described as a series of blocks of an algorithm, it is to be appreciated that the methodologies are not limited by the order of the blocks. Some blocks can occur in different orders and/or concurrently with other blocks from that shown and described. Moreover, less than all the illustrated blocks may be used to implement an example methodology. Blocks may be combined or separated into multiple actions/components. Furthermore, additional and/or alternative methodologies can employ additional actions that are not illustrated in blocks. The methods described herein are limited to statutory subject matter under 35 U.S.C § 101.

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

A “data structure”, as used herein, is an organization of data in a computing system that is stored in a memory, a storage device, or other computerized system. A data structure may be any one of, for example, a data field, a data file, a data array, a data record, a database, a data table, a graph, a tree, a linked list, and so on. A data structure may be formed from and contain many other data structures (e.g., a database includes many data records). Other examples of data structures are possible as well, in accordance with other embodiments.

“Computer-readable medium” or “computer storage medium”, as used herein, refers to a non-transitory medium that stores instructions and/or data configured to perform one or more of the disclosed functions when executed. Data may function as instructions in some embodiments. A computer-readable medium may take forms, including, but not limited to, non-volatile media, and volatile media. Non-volatile media may include, for example, optical disks, magnetic disks, and so on. Volatile media may include, for example, semiconductor memories, dynamic memory, and so on. Common forms of a computer-readable medium may include, but are not limited to, a floppy disk, a flexible disk, a hard disk, a magnetic tape, other magnetic medium, an application specific integrated circuit (ASIC), a programmable logic device, a compact disk (CD), other optical medium, a random access memory (RAM), a read only memory (ROM), a memory chip or card, a memory stick, solid state storage device (SSD), flash drive, and other media from which a computer, a processor or other electronic device can function with. Each type of media, if selected for implementation in one embodiment, may include stored instructions of an algorithm configured to perform one or more of the disclosed and/or claimed functions. Computer-readable media described herein are limited to statutory subject matter under 35 U.S.C § 101.

“Logic”, as used herein, represents a component that is implemented with computer or electrical hardware, a non-transitory medium with stored instructions of an executable application or program module, and/or combinations of these to perform any of the functions or actions as disclosed herein, and/or to cause a function or action from another logic, method, and/or system to be performed as disclosed herein. Equivalent logic may include firmware, a microprocessor programmed with an algorithm, a discrete logic (e.g., ASIC), at least one circuit, an analog circuit, a digital circuit, a programmed logic device, a memory device containing instructions of an algorithm, and so on, any of which may be configured to perform one or more of the disclosed functions. In one embodiment, logic may include one or more gates, combinations of gates, or other circuit components configured to perform one or more of the disclosed functions. Where multiple logics are described, it may be possible to incorporate the multiple logics into one logic. Similarly, where a single logic is described, it may be possible to distribute that single logic between multiple logics. In one embodiment, one or more of these logics are corresponding structure associated with performing the disclosed and/or claimed functions. Choice of which type of logic to implement may be based on desired system conditions or specifications. For example, if greater speed is a consideration, then hardware would be selected to implement functions. If a lower cost is a consideration, then stored instructions/executable application would be selected to implement the functions. Logic is limited to statutory subject matter under 35 U.S.C. § 101.

An “operable connection”, or a connection by which entities are “operably connected”, is one in which signals, physical communications, and/or logical communications may be sent and/or received. An operable connection may include a physical interface, an electrical interface, and/or a data interface. An operable connection may include differing combinations of interfaces and/or connections sufficient to allow operable control. For example, two entities can be operably connected to communicate signals to each other directly or through one or more intermediate entities (e.g., processor, operating system, logic, non-transitory computer-readable medium). Logical and/or physical communication channels can be used to create an operable connection.

“User”, as used herein, includes but is not limited to one or more persons, computers or other devices, or combinations of these.

While the disclosed embodiments have been illustrated and described in considerable detail, it is not the intention to restrict or in any way limit the scope of the appended claims to such detail. It is, of course, not possible to describe every conceivable combination of components or methodologies for purposes of describing the various aspects of the subject matter. Therefore, the disclosure is not limited to the specific details or the illustrative examples shown and described. Thus, this disclosure is intended to embrace alterations, modifications, and variations that fall within the scope of the appended claims, which satisfy the statutory subject matter requirements of 35 U.S.C. § 101.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

Therefore, provided herein is a new and improved system and method for managing medical devices and medical device consumables, which according to various embodiments of the present invention, offers the following advantages: ease of use; the ability to keep track of operating condition parameters in medical devices; the ability to automatically correct the operating condition parameter in the medical device without user intervention, if needed; the ability to provide feedback regarding the operating condition parameters in the medical device for wearer edification, preventative maintenance, and or product improvement; the ability to allow the user to pre-set levels of operating conditions within the hood; and the ability to provide audible and haptic (vibrate) alerts.

In fact, in many of the preferred embodiments, these advantages of ease of use, the ability to keep track of operating condition parameters in medical devices, the ability to automatically correct the operating condition parameter in the medical device without user intervention, if needed, the ability to provide feedback regarding the operating condition parameters in the medical device for wearer edification, preventative maintenance, and or product improvement, the ability to allow the user to pre-set levels of operating conditions within the hood, and the ability to provide audible and haptic (vibrate) alerts.

What is claimed is:

1. A system for managing operating condition parameters in a medical device, comprising:
a surgical gown;
a surgical hood operatively connected to the surgical gown, wherein the hood is configured to be located over a head and neck area of a wearer such that the head and neck area of the wearer are substantially enclosed within the hood;
a ventilation system located within the surgical gown and the surgical hood, wherein the ventilation system is configured to be retained by shoulders of the wearer of the ventilation system in order to provide ventilation air within the surgical gown and surgical hood; and
an operating parameter measurement assembly operatively connected to the ventilation system, wherein the operating parameter measurement assembly further comprises;
a plurality of operating condition parameter sensors located within the surgical hood, and
a mobile computing device which includes a display and an alarm and is configured with an application, wherein the mobile computing device is operatively connected to the plurality of operating condition parameter sensors, wherein the application is configured to allow the wearer to select at least one operating condition parameter preference within the hood while the wearer is wearing the hood during a medical procedure, and wherein the at least one operating condition parameter preference includes, but are not limited to, selected threshold levels of carbon dioxide (CO₂), temperature, humidity, oxygen (O₂), volatile organic compounds (VOCs), and/or air pressure.

2. The system, according to claim 1, wherein the ventilation system is further comprised of:
a fan motor;
an impeller connected to the fan motor; and
a motor sensor/wireless identification system connected to the fan motor, wherein the motor sensor/wireless identification system includes a printed circuit board and a tachometer, and wherein the printed circuit board is configured to interact with a processor to determine a speed at which the fan motor is operating.

3. The system, according to claim 1, wherein the operating parameter measurement assembly is further comprised of:
a microphone assembly located adjacent to the at least one of the plurality of operating condition parameter sensors.

4. The system, according to claim 1, wherein the plurality of operating condition parameter sensors is configured to be able to measure levels of carbon dioxide (CO₂), temperature, humidity, oxygen (O₂), VOCs, and/or air pressure) while the wearer is donning the hood and the ventilation system is operating.

5. The system, according to claim 1, wherein the application is configured to activate the alarm if a selected threshold level of carbon dioxide (CO₂), temperature, humidity, oxygen (O₂), volatile organic compounds (VOCs), and/or air pressure is exceeded.

6. The system, according to claim 3, wherein the application is configured to perform the following:
display on the mobile computing device a login and welcome home page;
display on the mobile computing device a settings page;
display on the mobile computing device a virtual assistant settings page;
display on the mobile computing device a system operation page;
display on the mobile computing device a first alert page; and
display on the mobile computing device a second alert page.

7. The system, according to claim 6, wherein the login and welcome home page is configured to display the following:
an average VOC level within the hood;
an average temperature level within the hood;
an average CO₂ level within the hood;
a next button; and
a setting page button.

8. The system, according to claim 6, wherein the settings page is configured to display the following:
a maximum allowed CO₂ setting that is configured to allow the wearer to select a desired allowed CO₂ level;
a maximum allowed temperature setting that is configured to allow the wearer to select a desired allowed temperature;
a CO₂ limit setting having a check box that is configured to allow the wearer to select if the wearer wants to receive a CO₂ alert through the alarm on the mobile computing device;
a temperature limit alert having a check box that is configured to allow the wearer to select if the wearer wants to receive a temperature alert through the alarm on the mobile computing device;
a virtual assistant voice recognition setting that is configured to allow the wearer to utilize the microphone assembly in conjunction with the mobile computing device and the application to control the system; and
a haptic feedback setting having a toggle button that is configured to allow the wearer to select if the wearer wants to receive a haptic feedback through the mobile computing device.

9. The system, according to claim 7, wherein the system operation page is configured to display the following:
the average VOC level within the hood;
the average temperature level within the hood;
the average CO₂ level within the hood;
the next page button;
the setting page button;
a day button;
a week button;
a month button;
a disconnect device button; and
a start surgery button.

10. The system, according to claim 8, wherein the first alert page is configured to display the following:
a first current CO₂ level within the hood, wherein the first current level of CO₂ exceeds the desired allowed CO₂ level;
a first alert that is configured to inform the wearer of a high CO₂ level;
a first current fan speed;
a first listing of operating condition parameter levels within the hood over a specified time interval during the medical procedure; and
an end surgery button.

11. The system, according to claim 10, wherein the second alert page is configured to display the following:
a second current CO₂ level within the hood, wherein the second current level of CO₂ does not exceed the desired allowed CO₂ level;
a second alert that is configured to inform the wearer of a return to the desired allowed CO₂ level;
a second current fan speed;
a second listing of operating condition parameter levels within the hood over a specified time interval during the medical procedure; and
the end surgery button.

12. A method of constructing a system for managing operating condition parameters in a medical device, the method comprising:
providing a surgical gown;
attaching a surgical hood to the surgical gown, wherein the hood is configured to be located over a head and neck area of a wearer such that the head and neck area of the wearer are substantially enclosed within the hood;
locating a ventilation system within the surgical gown and the surgical hood, wherein the ventilation system is configured to be retained by shoulders of the wearer of the ventilation system in order to provide ventilation air within the surgical gown and surgical hood; and
attaching an operating parameter measurement assembly to the ventilation system, wherein the operating parameter measurement assembly further comprises;
a plurality of operating condition parameter sensors located within the surgical hood, and
a mobile computing device which includes a display and an alarm and is configured with an application, wherein the mobile computing device is operatively connected to the plurality of operating condition parameter sensors, wherein the application is configured to allow the wearer to select at least one operating condition parameter preference within the hood while the wearer is wearing the hood during a medical procedure, and wherein the at least one operating condition parameter preference includes, but are not limited to, selected threshold levels of carbon dioxide (CO₂), temperature, humidity, oxygen (O₂), volatile organic compounds (VOCs), and/or air pressure.

13. The method, according to claim 12, wherein the ventilation system is further comprised of:
a fan motor;
an impeller connected to the fan motor; and
a motor sensor/wireless identification system connected to the fan motor,
wherein the motor sensor/wireless identification system includes a printed circuit board and a tachometer, and wherein the printed circuit board is configured to interact with a processor to determine a speed at which the fan motor is operating.

14. The method, according to claim 12, wherein the operating parameter measurement assembly is further comprised of:
a microphone assembly located adjacent to the at least one of the plurality of operating condition parameter sensors.

15. The method, according to claim 12, wherein the application is configured to activate the alarm if a selected threshold level of carbon dioxide (CO₂), temperature, humidity, oxygen (O₂), volatile organic compounds (VOCs), and/or air pressure is exceeded.

16. The method, according to claim 14, wherein the application is configured to perform the following:
display on the mobile computing device a login and welcome home page;
display on the mobile computing device a settings page;
display on the mobile computing device a virtual assistant settings page;
display on the mobile computing device a system operation page;
display on the mobile computing device a first alert page; and
display on the mobile computing device a second alert page.

17. The method, according to claim 16, wherein the login and welcome home page is configured to display the following:
an average VOC level within the hood;
an average temperature level within the hood;
an average CO₂ level within the hood;
a next button; and
a setting page button.

18. The method, according to claim 16, wherein the settings page is configured to display the following:
a maximum allowed CO₂ setting that is configured to allow the wearer to select a desired allowed CO₂ level;
a maximum allowed temperature setting that is configured to allow the wearer to select a desired allowed temperature;
a CO₂ limit setting having a check box that is configured to allow the wearer to select if the wearer wants to receive a CO₂ alert through the alarm on the mobile computing device;
a temperature limit alert having a check box that is configured to allow the wearer to select if the wearer wants to receive a temperature alert through the alarm on the mobile computing device;
a virtual assistant voice recognition setting that is configured to allow the wearer to utilize the microphone assembly in conjunction with the mobile computing device and the application to control the system; and
a haptic feedback setting having a toggle button that is configured to allow the wearer to select if the wearer wants to receive a haptic feedback through the mobile computing device.

19. The method, according to claim 17, wherein the system operation page is configured to display the following:
the average VOC level within the hood;
the average temperature level within the hood;
the average CO₂ level within the hood;
the next page button;
the setting page button;
a day button;
a week button;
a month button;
a disconnect device button; and
a start surgery button.

20. The method, according to claim 18, wherein the first alert page is configured to display the following:
a first current CO₂ level within the hood, wherein the first current level of CO₂ exceeds the desired allowed CO₂ level;
a first alert that is configured to inform the wearer of a high CO₂ level;
a first current fan speed;
a first listing of operating condition parameter levels within the hood over a specified time interval during the medical procedure; and
an end surgery button.
